(19) United States
(12) Patent Application Publication
(10) Pub. No.: US 2025/0177009 A1
(43) Pub. Date: Jun. 5, 2025

(54) CONNECTOR ROD FOR CORRECTION OF SPINAL DEFORMITY

(71) Applicant: The General Hospital Corporation, Boston, MA (US)

(72) Inventor: Saechin Kim, Andover, MA (US)

(73) Assignee: The General Hospital Corporation, Boston, MA (US)

(21) Appl. No.: 18/686,018

(22) PCT Filed: Aug. 24, 2022
(86) PCT No.: PCT/US2022/041340

§ 371 Date: Feb. 23, 2024

Related U.S. Application Data
(60) Provisional application No. 63/236,652, filed on Aug. 24, 2021.

(51) Int. Cl. A61B 17/70; A61B 17/00

(52) U.S. Cl. CPC A61B 17/7023; A61B 17/7034; A61B 17/7043; A61B 17/7053; A61B 2017/00407

(57) ABSTRACT

A bone surgical apparatus provides an improved method and apparatus to manipulate the curvature of the spine. The bone surgical apparatus can include a connector comprising a rod having a first end section and a second end section wherein the rod is dimensioned to connect a pair of pedicle screws, and a fastener attached to the rod wherein the fastener comprises a head including spaced apart walls defining a recess for receiving an alignment rod.

### CROSS-REFERENCE TO RELATED APPLICATIONS

This application is based on, claims benefit of, and claims priority to U.S. Application No. 63/236,652 filed on Aug. 24, 2021, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

This invention relates to spinal surgery methods and associated apparatus.

### 2. Description of the Related Art

Spinal surgery for spinal deformities, such as scoliosis and kyphosis, is often performed on children who have not finished growing. Scoliosis surgery usually includes correction of the deformity, and a variety of techniques are used to correct the curve. In the majority of cases, the surgery is done using the posterior approach by fixing either pedicle screws or hooks, that is fixation devices, into the vertebrae. These fixation devices are used to correct the spinal deformity by either: (1) using precontoured rod(s) to reduce the spinal deformity to the contoured rod through the fixation devices; or (2) bending the rod(s) to fit into the fixation devices and then correct the curve by rotating the rod(s) and/or rotating the spine around the rod(s) and/or distraction/compression between the fixation devices along the rod(s); or (3) a combination of the above two techniques. In all these cases, the fixation needs to span from above the deformity to below the deformity.

For curves that are primarily in the lumbar spine, an anterior approach has been used to directly derotate the apex of the curve by first inserting vertebral screws into the vertebral body, putting in a contoured rod into the tulip shaped heads of the screws, and then rotating the rod in the tulip shaped heads of the screws.

The anterior approach does not need fixation above and below the deformity and therefore, the length of the fusion would be shorter than if the posterior methods discussed above are used. However, the anterior approach has its issues including limited number of levels that can be instrumented from a single approach, and the complications associated with the anterior approach such as intercostal neuritis and abdominal hernia.

Therefore, what is needed is an improved method and apparatus to manipulate the curvature of the spine.

### SUMMARY OF THE INVENTION

The present invention meets the foregoing needs. In one aspect, the disclosure provides a bone surgical apparatus for manipulating a curvature of a spine. The bone surgical apparatus comprises a connector comprising a rod having a first end section and a second end section wherein the rod is dimensioned to connect a pair of pedicle screws, and a fastener attached to the rod wherein the fastener comprises a head including spaced apart walls defining a recess for receiving an alignment rod. In one embodiment, the fastener is located at the first end section of the rod. In one embodiment, the fastener is located between the first end section and the second end section of the rod.

In one embodiment, an additional fastener is attached to the rod wherein the additional fastener comprises a head including spaced apart walls defining a recess for receiving an alignment rod. The fastener can be located at the first end section of the rod, and the additional fastener can be located at the second end section of the rod.

The bone surgical apparatus can further comprise a plurality of additional fasteners attached to the rod, wherein at least one of the additional fasteners comprises a head including spaced apart walls defining a recess for receiving an alignment rod.

The bone surgical apparatus can further comprise a second fastener attached to the rod wherein the second fastener comprises a head including spaced apart walls defining a recess for receiving an alignment rod, and a third fastener attached to the rod wherein the third fastener comprises a head including spaced apart walls defining a recess for receiving an alignment rod, wherein the fastener is located at the first end section of the rod, wherein the second fastener is located at the second end section of the rod, and wherein the third fastener is located between the first end section and the second end section of the rod.

In one embodiment of the bone surgical apparatus, a longitudinal axis of the head of the fastener is polyaxial with respect to a longitudinal axis of the rod.

The bone surgical apparatus can further comprise a pair of pedicle screws configured to attach to a vertebra and dimensioned to receive the rod of the connector.

The bone surgical apparatus can further comprise a first pair of pedicle screws configured to attach to a first vertebra, the first pair of pedicle screws being attached to the rod of the connector, a second connector comprising a second rod having a first end section and a second end section wherein the second rod is dimensioned to connect a pair of pedicle screws, and a second fastener attached to the second rod wherein the second fastener comprises a head including spaced apart walls defining a recess for receiving the alignment rod, and a second pair of pedicle screws configured to attach to a second vertebra, the second pair of pedicle screws being attached to the second rod of the second connector.

The bone surgical apparatus can further comprise an alignment rod connected to the fastener of the rod and the second fastener of the second rod. In one embodiment, the fastener is located between the first end section and the second end section of the rod, and the second fastener is located between the first end section and the second end section of the second rod.

The bone surgical apparatus can further comprise a first additional fastener attached to the first end section of the rod. The bone surgical apparatus can further comprise a second additional fastener attached to the second end section of the rod. The bone surgical apparatus can further comprise a second alignment rod connected to the first additional fastener and the second additional fastener, and a third alignment rod connected to the first additional fastener and the second additional fastener.

In one embodiment, the fastener is located at the first end section of the rod, and the second fastener is located at the first end section of the second rod.

The bone surgical apparatus can further comprise a first additional fastener attached to the second end section of the rod, a second additional fastener attached to the second end section of the second rod, and a second alignment rod connected to the first additional fastener and the second additional fastener. In one embodiment, the first additional fastener comprises a head including spaced apart walls defining a recess for receiving the second alignment rod, and the second additional fastener comprises a head including spaced apart walls defining a recess for receiving the second alignment rod.

The bone surgical apparatus can further comprise a first pair of pedicle screws configured to attach to a first vertebra, the first pair of pedicle screws being attached to the rod of the connector; an additional pedicle screw configured to attach to a second vertebra, the additional pedicle screw being connected to a fusion rod; and an angled rod, wherein the fastener is located at the first end section of the rod, and wherein the angled rod is connected to the fastener and the fusion rod.

The bone surgical apparatus can further comprise a first pair of pedicle screws configured to attach to a first vertebra, the first pair of pedicle screws being attached to the rod of the connector; an additional pedicle screw configured to attach to a second vertebra, the additional pedicle screw being connected to a fusion rod; and an angled rod, wherein the fastener is located at the first end section of the rod, and wherein the angled rod is connected to the fastener and the fusion rod.

In one embodiment, the first end section of the rod and the second end section of the rod are connected by a hinge. A first section of the hinge can be attached to the first end section of the rod, a second section of the hinge can be attached to the second end section of the rod, and the hinge can include a locking device for locking the hinge in one of a plurality of relative positions between the first section of the hinge and the second section of the hinge.

In one embodiment, the first end section of the rod and the second end section of the rod are connected by an adjustment mechanism structured to retain the first end section of the rod and the second end section of the rod at any of a plurality of distances between the first end section of the rod and the second end section of the rod thereby providing for variable lengths of the rod. The adjustment mechanism can be connected by a hinge to at least one of the first end section of the rod and the second end section of the rod.

The bone surgical apparatus can further comprise a first pedicle screw and a second pedicle screw configured to attach to a first vertebra, the rod of the connector being attached to the first pedicle screw, the fastener being located at the first end section of the rod; a third pedicle screw and a fourth pedicle screw configured to attach to a second vertebra; and a fixator having a first end section and a second end section, the first end section of the fixator being attached to the fastener and the second pedicle screw, and the second end section of the fixator being attached to the fourth pedicle screw. In one embodiment, the first end section of the fixator comprises an alignment rod, a first end of the alignment rod is received in the recess of the fastener for attachment to the fastener, and a second end of the alignment rod is received in a recess in a head of the second pedicle screw for attachment to the second pedicle screw. A method for manipulating a curvature of a spine of a subject can include the steps of (i) surgically implanting the bone surgical apparatus into a subject such that the first pedicle screw and the second pedicle screw are attached to the first vertebra and the third pedicle screw and the fourth pedicle screw are attached to the second vertebra; (ii) moving the fixator to achieve a desired alignment of the first vertebra and the second vertebra; (iii) removing the connector from the first pedicle screw; and (iv) attaching an alignment rod to the first pedicle screw and the third pedicle screw.

In another aspect, the disclosure provides a bone surgical apparatus for manipulating a curvature of a spine. The bone surgical apparatus comprises a connector comprising a rod having a first end section and a second end section wherein the rod is dimensioned to connect a pair of pedicle screws, and a fastener attached to the rod wherein the fastener comprises sections for clamping a band. In one embodiment, the fastener is located at the first end section of the rod.

The bone surgical apparatus can further comprise a second fastener attached to the rod wherein the second fastener comprises sections for clamping a second band, wherein the fastener is located at the first end section of the rod, wherein the second fastener is located between the first end section and the second end section of the rod.

The bone surgical apparatus can further comprise a first pair of pedicle screws configured to attach to a first vertebra, the first pair of pedicle screws being attached to the rod of the connector, a second connector comprising a second rod having a first end section and a second end section wherein the second rod is dimensioned to connect a pair of pedicle screws, and a second fastener attached to the second rod, wherein the second fastener comprises a sections for clamping a band; a second pair of pedicle screws configured to attach to a second vertebra, the second pair of pedicle screws being attached to the second rod of the second connector, and a band connected to the fastener and the second fastener. In one embodiment, a first additional fastener attached to the rod between the first end section and the second end section of the rod; a second additional fastener attached to the second rod between the first end section and the second end section of the second rod; a second band connected to the first additional fastener and the second additional fastener; and a sheath surrounding the second band.

In another aspect, the disclosure provides a bone surgical apparatus for manipulating a curvature of a spine. The bone surgical apparatus comprises a connector comprising a rod having a first end section and a second end section wherein the rod is dimensioned to connect a pair of pedicle screws, and a latch attached to the first end section of the rod wherein the latch comprises a first wall and a second wall defining an interior region for receiving a portion of a loop. In one embodiment, an additional latch is attached to the second end section of the rod wherein the additional latch comprises a first wall and a second wall defining an interior region for receiving a portion of a loop. In one embodiment, the first wall has a first terminal end, the second wall has a second terminal end, the first terminal end and the second terminal end are positioned to define a space between the first terminal end and the second terminal end, the latch includes a movable barrier for opening and closing the space between the first terminal end and the second terminal end, the portion of the loop is received in the interior region when the space is open, and the portion of the loop is retained in the interior region when the space is closed.

The bone surgical apparatus can further comprise a first pair of pedicle screws configured to attach to a first vertebra, the first pair of pedicle screws being attached to the rod of the connector, a second connector comprising a second rod having a first end section and a second end section wherein the second rod is dimensioned to connect a pair of pedicle screws, and a second latch attached to the first end section of the second rod wherein the second latch comprises a first wall and a second wall defining an interior region for receiving a portion of the loop, a second pair of pedicle screws configured to attach to a second vertebra, the second pair of pedicle screws being attached to the second rod of the second connector, and a loop having a first portion retained in the interior region of the latch and a second portion retained in the interior region of the second latch. In one embodiment, the loop is dimensioned such that the loop is tensioned when retained in the interior region of the latch and retained in the interior region of the second latch.

The bone surgical apparatus can further comprise a first additional latch attached to the second end section of the rod, wherein the first additional latch comprises a first wall and a second wall defining an interior region for receiving a portion of a loop, a second additional latch attached to the second end section of the second rod, wherein the second additional latch comprises a first wall and a second wall defining an interior region for receiving a portion of a loop, and a second loop having a first portion retained in the interior region of the first additional latch and a second portion retained in the interior region of the second additional latch.

The bone surgical apparatus can further comprise a first pair of pedicle screws configured to attach to a first vertebra, the first pair of pedicle screws being attached to the rod of the connector; a second connector comprising a second rod having a first end section and a second end section wherein the second rod is dimensioned to connect a pair of pedicle screws, and a fastener attached to the second rod wherein the fastener comprises a head including spaced apart walls defining a recess for receiving a rod; a second pair of pedicle screws configured to attach to a second vertebra, the second pair of pedicle screws being attached to the second rod of the second connector; a third connector comprising a third rod having a first end section and a second end section wherein the third rod is dimensioned to connect a pair of pedicle screws, and a second latch attached to the first end section of the third rod wherein the second latch comprises a first wall and a second wall defining an interior region for receiving a portion of the loop, the third rod being attached to the fastener of the second connector; and a loop having a first portion retained in the interior region of the latch and a second portion retained in the interior region of the second latch. In one embodiment, the loop is formed from a band of material. In one embodiment, the loop is formed from a tubular material.

The bone surgical apparatus can further comprise a fastener attached to the second end section of the rod wherein the fastener comprises a head including spaced apart walls defining a recess for receiving an alignment rod.

The bone surgical apparatus can further comprise a fastener attached to the rod between the first end section and the second end section, wherein the fastener comprises a head including spaced apart walls defining a recess for receiving an alignment rod.

In one embodiment, the first end section of the rod and the second end section of the rod are connected by a hinge. A first section of the hinge can be attached to the first end section of the rod, a second section of the hinge can be attached to the second end section of the rod, and the hinge can include a locking device for locking the hinge in one of a plurality of relative positions between the first section of the hinge and the second section of the hinge.

In one embodiment, the first end section of the rod and the second end section of the rod are connected by an adjustment mechanism structured to retain the first end section of the rod and the second end section of the rod at any of a plurality of distances between the first end section of the rod and the second end section of the rod thereby providing for variable lengths of the rod. In one embodiment, the adjustment mechanism is connected by a hinge to at least one of the first end section of the rod and the second end section of the rod.

In another aspect, the disclosure provides a bone surgical apparatus for manipulating a curvature of a spine. The bone surgical apparatus comprises a latch connector comprising a rod adapter dimensioned to attach to a fusion rod, a central section attached to the rod adapter wherein the central section is dimensioned to attach to a head of a pedicle screw, and a latch attached to the central section wherein the latch comprises a first wall and a second wall defining an interior region for receiving a portion of a loop. In one embodiment, the first wall has a first terminal end, the second wall has a second terminal end, the first terminal end and the second terminal end are positioned to define a space between the first terminal end and the second terminal end, the latch includes a movable barrier for opening and closing the space between the first terminal end and the second terminal end, the portion of the loop is received in the interior region when the space is open, and the portion of the loop is retained in the interior region when the space is closed.

The bone surgical apparatus can further comprise a fusion rod; a first connector comprising a first rod having a first end section and a second end section wherein the first rod is dimensioned to connect a pair of pedicle screws, and a first fastener attached to the first rod wherein the first fastener comprises a first head including spaced apart walls defining a first recess, the fusion rod being positioned in the first recess and being connected to the first head; a first pair of pedicle screws configured to attach to a first vertebra, the first pair of pedicle screws being attached to the first rod of the first connector; a second connector comprising a second rod having a first end section and a second end section wherein the second rod is dimensioned to connect a pair of pedicle screws, and a second fastener attached to the second rod wherein the second fastener comprises a second head including spaced apart walls defining a second recess, the fusion rod being positioned in the second recess and being connected to the second head; a second pair of pedicle screws configured to attach to a second vertebra, the second pair of pedicle screws being attached to the second rod of the second connector; a third connector a third rod having a first end section and a second end section wherein the third rod is dimensioned to connect a pair of pedicle screws, and a second latch attached to the first end section of the third rod wherein the second latch comprises a first wall and a second wall defining an interior region for receiving a portion of the loop; a third pair of pedicle screws configured to attach to a third vertebra, the third pair of pedicle screws being attached to the third rod of the third connector; and a loop having a first portion retained in the interior region of the latch and a second portion retained in the interior region of the second latch, wherein the rod adapter of the latch connector is attached to the fusion rod, and the central section of the latch connector is attached to the second head of the second fastener.

In one embodiment, the rod adapter of the latch connector is attached to the fusion rod by a set screw. In one embodiment, the central section of the latch connector surrounds the second head of the second fastener to engage the second head of the second fastener when the latch connector is attached to the second head of the second fastener.

In another aspect, the disclosure provides a bone surgical apparatus for manipulating a curvature of a spine. The bone surgical apparatus comprises an adjustment mechanism comprising a first rod section having an inner end and an outer end, a second rod section having an inner end and an outer end having an outer surface shape, and a hinge attached to the inner end of the first rod section and to the inner end of the second rod section, wherein the outer end of the first rod section comprises a wall defining a recess dimensioned for receiving a rod end section having a perimeter shaped the same as the outer surface shape of the outer end of the second rod section. A first section of the hinge can be attached to the inner end of the first rod section, a second section of the hinge can be attached to the inner end of the second rod section, and the hinge can include a locking device for locking the hinge in one of a plurality of relative positions between the first section of the hinge and the second section of the hinge.

The bone surgical apparatus can further comprise a second adjustment mechanism comprising a third rod section having an inner end and an outer end, a fourth rod section having an inner end and an outer end having an outer surface shape, and a second hinge attached to the inner end of the third rod section and to the inner end of the fourth rod section, wherein the outer end of the third rod section comprises a wall defining a recess dimensioned for receiving the outer end of the second rod section. The hinge can include a ratchet for controlling motion in both of a first rotational direction and an opposite second rotational direction of the hinge.

The bone surgical apparatus can further comprise a first pedicle screw configured to attach to a first vertebra, the first rod section being attached to the first pedicle screw; and a second pedicle screw configured to attach to a second vertebra, the fourth rod section being attached to the second pedicle screw, wherein the outer end of the second rod section is received in the recess in the outer end of the third rod section. In one embodiment, the outer end of the second rod section is secured in the recess in the outer end of the third rod section by a set screw. In one embodiment, the outer end of the second rod section is secured in the recess in the outer end of the third rod section by an interference fit. In one embodiment, a first section of the second hinge is attached to the inner end of the third rod section, a second section of the second hinge is attached to the inner end of the fourth rod section, and the second hinge includes a locking device for locking the second hinge in one of a plurality of relative positions of the second hinge between the first section of the second hinge and the second section of the second hinge. In one embodiment, the hinge is locked in one of the plurality of relative positions of the hinge by a first set screw, and the second hinge is locked in one of the plurality of relative positions of the second hinge by a second set screw.

The bone surgical apparatus can further comprise a fusion rod; a first pedicle screw configured to attach to a first vertebra, the fusion rod being attached to the first pedicle screw; a second pedicle screw configured to attach to a second vertebra, the second rod section being attached to the second pedicle screw, wherein an end of the fusion rod is received in the recess in the outer end of the first rod section. In one embodiment, the end of the fusion rod is secured in the recess in the outer end of the first rod section by a set screw.

In another aspect, the disclosure provides a bone surgical apparatus for manipulating a curvature of a spine. The bone surgical apparatus comprises a first pedicle screw and a second pedicle screw configured to attach to a first vertebra; a third pedicle screw and a fourth pedicle screw configured to attach to a second vertebra; and a fixator having a first end section and a second end section, the first end section of the fixator including a hinged rod attached to the first pedicle screw, and the second end section of the fixator being attached to the fourth pedicle screw. In one embodiment, a first end of the hinged end is received in a recess in a head of the first pedicle screw for attachment to the first pedicle screw, a second end of the hinged end is received in a recess in a head of the second pedicle screw for attachment to the second pedicle screw and the second end section of the fixator is received in a recess in a head of the fourth pedicle screw for attachment to the fourth pedicle screw. A method for manipulating a curvature of a spine of a subject can include the steps of (i) surgically implanting the bone surgical apparatus into a subject such that the first pedicle screw and the second pedicle screw are attached to the first vertebra and the third pedicle screw and the fourth pedicle screw are attached to the second vertebra; (ii) moving the fixator to achieve a desired alignment of the first vertebra and the second vertebra; (iii) removing the hinged end from the recess in the head of the first pedicle screw; and (iv) attaching an alignment rod to the first pedicle screw and the third pedicle screw.

The present disclosure provides a bone surgical apparatus with or without a hinge/ratchet that can be used to correct spinal deformity with or without spinal fusion.

The present disclosure provides an approach using devices that use intravertebral inter-pedicle-screw rod(s) with fasteners (that can have a tulip-shaped section) at the ends such that short fusion can be done using the posterior approach without the complications associated with the anterior approach. In this approach, each vertebra at the apex of the deformity is individually fixed to pedicle screws and the device is placed into the two pedicle screws of each vertebra. Then each vertebra is directly manipulated using a combination of rotational, compression, and distraction forces such that the curve is corrected. The correction does not require precontoured rod(s) between the vertebrae. However, once the correction is achieved, a precontoured rod can be used to hold the curve correction.

If the same correction forces, such as rotational forces, are applied directly to the pedicle screws, then the screw would just rotate in the pedicle and the vertebra would not be manipulated. Rotational forces would cause the hooks to move or come out of their insertion sites. Therefore, direct manipulation of each vertebra requires one or more devices of the invention.

Another benefit of the device is that corrective forces are applied and the stress of the forces would be shared by the two pedicle screws in the same vertebra. Therefore, greater corrective forces can be applied.

Another benefit is that since each vertebra is manipulated directly, one can also directly adjust lordosis and kyphosis.

In certain embodiments, the connector rod can be a straight rod, or have a connector in the middle to allow for variable length of the rod or have a hinge within the rod or a combination of variable length as well as a hinge.

This process, which can be referred to Direct Vertebral ReAlignment (DVRA), allows surgeons to directly address the spinal deformity at its apex without extending the spinal construct to above and below the deformity. Therefore, shorter fusions can be done. For example, one of the more common fusions for adolescent idiopathic scoliosis is done from T4 to L1 for a total of ten levels. Typically, this type of curve can be corrected by manipulating and fusing four to seven vertebrae.

This is even more important in very young children who need “growing” rods for their scoliosis. Most current “growing rods” rely on distraction across the deformity with instruments above and below the deformity. Using DVRA, the surgeon can directly manipulate the apex of the curve and allow the rest of the spine to grow. The concept is similar to the Shilla technique except in the Shilla technique, the apex is fused and the rest of the spine is allowed to “grow” over the two rods. In this method, the growth is not inhibited by or limited to the rods.

The present invention provides a method and apparatus that overcome the aforementioned issues by directly correcting the curve of the spine by tethering of the convexity of the curve rather than distracting the spine. Scoliosis surgery often includes correction of the deformity and a variety of techniques are used to correct the curve. In majority of cases, they can be divided into two categories: one is to use precontoured rod(s) to reduce the spinal curvature to the contoured rod; and the second is to stabilize the curve above and below the curve with contoured rod(s) and then correct the curve by rotating the rod(s) and/or rotating the spine around the rod(s). In both of these cases, the fixation needs to span from above the deformity to below the deformity.

For curves that are primarily in the lumbar spine, an anterior approach can be used to directly derotate the apex of the curve by first inserting vertebral screws, putting in a contoured rod into the fastener heads of the screws and then rotating it in the fastener heads of the screws. In all these cases, the correction involves using precontoured rods and/or fixation that spans to above and below the deformity. In addition, in the anterior approach has its issues including limited number of levels that can be instrumented from a single approach.

In the approach of the present invention, the apex of the deformity is directly re-aligned without relying on a contoured rod and using a posterior approach. The reason the rod is necessary in the prior approaches discussed above is that trying to manipulate the vertebrae through the screws without the rod would result in the pedicle screws turning rather than correction of the curve. To directly realign the vertebrae, the solution is to place a rod between a pair of pedicle screws within the same vertebra, that is between the screws in the right and left pedicle of a single vertebra. Then using the devices of the present invention that are used to derotate the vertebra, each vertebra can be directly realigned. Once realigned, an external fixation system that can clamp on the rod can be used to hold the desired alignment.

A next step may be to free up the fastener heads of the pedicle screws on one side so that a rod can be placed for the fusion. In one version of this invention, the rod has a hinge so that it can bend in one plane. The pedicle screws in the same vertebra tend to converge and so the tulip shaped heads are not colinear. The hinged rod can be bent so that the rods on the two sides of the fastener head each can fit exactly into each fastener head; this is much more difficult with solid rods when non-polyaxial screws are used.

If the hinge is aligned so that the rod can bend in a plane parallel to the fastener head of the pedicle screw, then, once the external fixation is applied to the rod on the one side of the hinge (called the first side) to hold the alignment in place, then the set caps on the other side (called the second side) can be removed and the rods can be bent at the hinges so that they are out of the tulip shaped heads of the second side. Then a rod can be placed into the fastener heads on the second side and stabilized with set caps. Then the external fixator can be removed followed by the set caps on first side followed by removal of the hinged rods. Then another rod can be placed into the empty tulip shaped heads of the first side to complete the instrumentation.

Hinged rods of the present invention can also be used for correction of spinal deformity including scoliosis and kyphosis. A rod with multiple hinges can be used to reduce the scoliosis as well as it can provide rotational force to correct the rotational deformity.

Scoliosis is a complex rotational deformity and so these hinged rods can be designed with a male component on one end and female component on the other end so that they can be interlinked and so that the male component can freely rotate until locked in placed using set screws. This would allow for multiplanar correction of the spinal deformity scoliosis. In a similar fashion, kyphosis can be corrected as well.

These and other features, aspects and advantages of various embodiments of the present invention will become better understood with regard to the following description, appended claims and accompanying Figures.

### BRIEF DESCRIPTION OF THE DRAWINGS

FIG. 1 illustrates schematically a scoliotic spine, viewed from the posterior.

FIG. 2 is a perspective view showing how a connector rod can be attached to a pedicle screw in a bone surgical apparatus according to the invention.

FIG. 3 is an axial view of a pedicle screw implanted in a vertebra.

FIG. 4 is a perspective view of a pedicle screw suitable for use in a bone surgical apparatus according to the invention.

FIG. 5 is a perspective view of one embodiment of an IntraVertebral InterPedicle (IVIP) rod suitable for use in a bone surgical apparatus according to the invention.

FIG. 6 is a cross-sectional view of a the rod of FIG. 5.

FIG. 7A is a posterior view of the rod of FIG. 5 connected to a pair of pedicle screws in another embodiment of a bone surgical apparatus according to the invention.

FIG. 7B is a posterior view showing assembly of one embodiment of a bone surgical apparatus according to the invention.

FIG. 7C is a partial cross-sectional view of the end section of the rod of FIG. 5 showing how an alignment rod can be attached to a fastener of the rod of FIG. 5 in a bone surgical apparatus according to the invention.

FIG. 7D is a posterior view showing assembly of another embodiment of a bone surgical apparatus according to the invention.

FIG. 8A is a perspective view of another embodiment of an IVIP rod suitable for use in a bone surgical apparatus according to the invention.

FIG. 8B is a perspective view of another embodiment of an IVIP rod suitable for use in a bone surgical apparatus according to the invention.

FIG. 8C is a perspective view of another embodiment of an IVIP rod suitable for use in a bone surgical apparatus according to the invention.

FIG. 9A is a perspective view of another embodiment of an IVIP rod suitable for use in a bone surgical apparatus according to the invention.

FIG. 9B is a posterior view showing assembly of another embodiment of a bone surgical apparatus according to the invention.

FIG. 10A is a perspective view of another embodiment of an IVIP rod suitable for use in a bone surgical apparatus according to the invention.

FIG. 10B is a posterior view of the rod of FIG. 10A connected to a pair of pedicle screws in another embodiment of a bone surgical apparatus according to the invention.

FIG. 11A is a perspective view of another embodiment of an IVIP rod suitable for use in a bone surgical apparatus according to the invention.

FIG. 11B is a posterior view of the rods of FIGS. 9A and 11A connected to pairs of pedicle screws on adjacent vertebrae in another embodiment of a bone surgical apparatus according to the invention.

FIG. 12A is a side view of another embodiment of an IVIP rod suitable for use in a bone surgical apparatus according to the invention.

FIG. 12B is a posterior view of the rod of FIG. 12A connected to pairs of pedicle screws in another embodiment of a bone surgical apparatus according to the invention.

FIG. 12C is a posterior view of the rod of FIG. 12A connected to pairs of pedicle screws on adjacent vertebrae in another embodiment of a bone surgical apparatus according to the invention.

FIG. 13A is a posterior view of another embodiment of a rod connected to pairs of pedicle screws on adjacent vertebrae in another embodiment of a bone surgical apparatus according to the invention.

FIG. 13B is a view taken along line 13B-13B of FIG. 13A of the rod of FIG. 13A with the latch in a closed position for retaining a loop.

FIG. 13C is a view taken along line 13B-13B of FIG. 13A of the rod of FIG. 13A with the latch in an open position for receiving a loop.

FIG. 14A is a perspective view of a loop suitable for use with the bone surgical apparatus of FIG. 13A.

FIG. 14B is a perspective view of another loop suitable for use with the bone surgical apparatus of FIG. 13A.

FIG. 15 is a posterior view of the loop of FIG. 14A retained by the bone surgical apparatus of FIG. 13A before manipulation of adjacent vertebrae.

FIG. 16 is a posterior view of the loop of FIG. 14A retained by the bone surgical apparatus of FIG. 13A after manipulation of adjacent vertebrae.

FIG. 17 is a posterior view of yet another embodiment of a rod connected to pairs of pedicle screws on adjacent vertebrae in another embodiment of a bone surgical apparatus according to the invention.

FIG. 18 is a posterior view of the loop of FIG. 14A retained by the bone surgical apparatus of FIG. 17 before manipulation of adjacent vertebrae.

FIG. 19 is a posterior view of the loop of FIG. 14A retained by the bone surgical apparatus of FIG. 17 after manipulation of adjacent vertebrae.

FIG. 20 is a posterior view of another embodiments of rods connected to pairs of pedicle screws on adjacent vertebrae in another embodiment of a bone surgical apparatus according to the invention.

FIG. 21 is a posterior view of an embodiment of a latch connector according to the invention.

FIG. 22 is a posterior view of the latch connector of FIG. 21 used in a bone surgical apparatus according to the invention.

FIG. 23 is a posterior view of other embodiments of a rod suitable for use in a bone surgical apparatus according to the invention.

FIG. 24 is a posterior view showing a bone surgical apparatus.

FIG. 25 is a posterior view showing embodiments of an adjustment mechanism of a bone surgical apparatus according to the invention.

FIG. 26 is a posterior view showing another embodiment of adjustment mechanisms connected to a pair of pedicle screws in another embodiment of a bone surgical apparatus according to the invention.

FIG. 27 is a posterior view showing another embodiment of a bone surgical apparatus according to the invention.

FIG. 28 is a posterior view showing another embodiment of a bone surgical apparatus according to the invention.

Like reference numerals will be used to refer to like parts from Figure to Figure in the following detailed description.

### DETAILED DESCRIPTION OF THE INVENTION

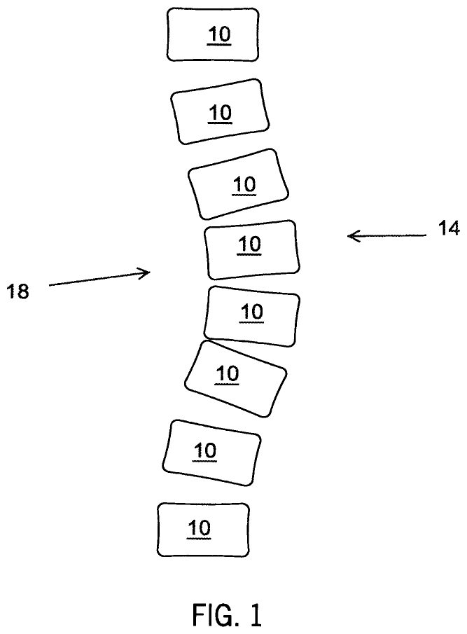

Embodiments of the invention may further be understood with reference to the Figures. Referring to FIG. 1, an illustration is shown of the anatomical situation of a typical scoliotic spine, in which some vertebrae 10 are displaced from a normal position at least in a lateral direction. FIG. 1 is a view from the posterior, and in this illustration, the vertebrae 10 are displaced to the patient's right. At the extreme of the abnormal displacement, there is, as labeled in FIG. 1, a convex portion 14 and a concave portion 18 opposed to the convex portion 14.

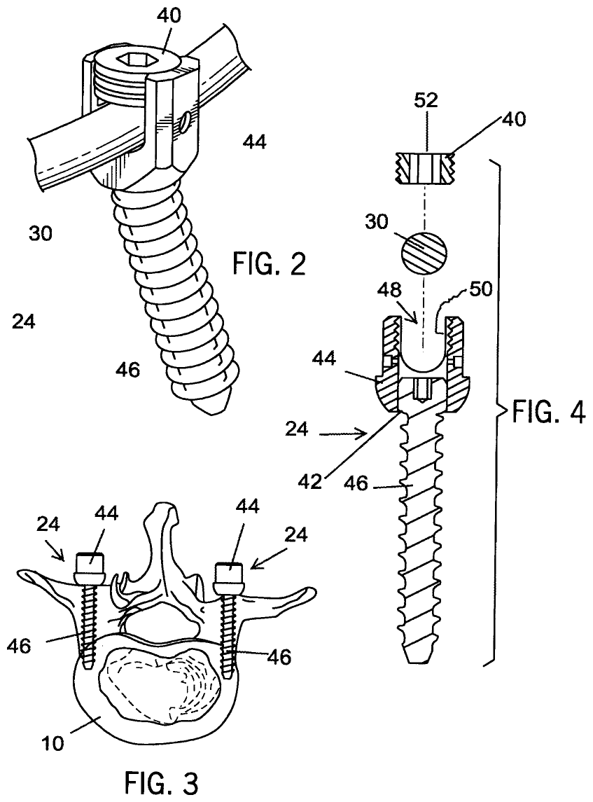

Referring to FIGS. 2, 3, and 4, one or more of a pedicle screw 24 can couple to the rod 30 of a bone surgical apparatus according to the invention to a vertebrae 10 of the spine. As illustrated in FIG. 2, the cylindrical rod 30 is receivable in a pedicle screw 24. In one example embodiment, as shown in FIGS. 2 and 4, the pedicle screws 24 are any of the various pedicle screws known in the art.

FIGS. 2 and 4 show a typical polyaxial pedicle screw 24 coupled with the cylindrical rod 30. In such a pedicle screw 24, when the set screw 40 presses against the cylindrical rod 30, the cylindrical rod 30 in turn presses on the at least partially spherical screw head 42 which in turn presses the screw head 42 against the interior surface of the polyaxial head 44, thereby locking the position of the screw shaft 46 with respect to the polyaxial head 44. More particularly, this locks all of the components involved, against all degrees of freedom of motion.

With continued reference to FIGS. 2-4, the pedicle screw 24 may be installed by inserting the screw shaft 46 through the top of the polyaxial head 44 and then driving screw shaft 46 into vertebrae 10 utilizing recess 48. Next, the cylindrical rod 30 can be positioned in the polyaxial head 44 and the set screw 40 may then be received in threaded section 50 to thereby vertically restrain screw head 42 and polyaxial head 44. The set screw 40 can be driven into the polyaxial head 44 utilizing recess 52.

Yet other embodiments of the present disclosure include pedicle screws that enable essentially only one degree of freedom of motion with respect to the screw shaft, which is a more restricted form of motion. In still other embodiments, the pedicle screw comprises a screw and head formed as a monolithic component. In this case, the head is no longer polyaxial, but is rather fixed relative to the screw head and shaft. The head of the pedicle screw may also be convertible from polyaxial to monoaxial using structures described in PCT Patent Application Publication No. WO 2017/035186. Although various structures and methods for restraining vertical movement of a polyaxial head that is separate from a screw head have been described herein, it is understood that additional or alternative structures and/or methods may be utilized in alternative embodiments.

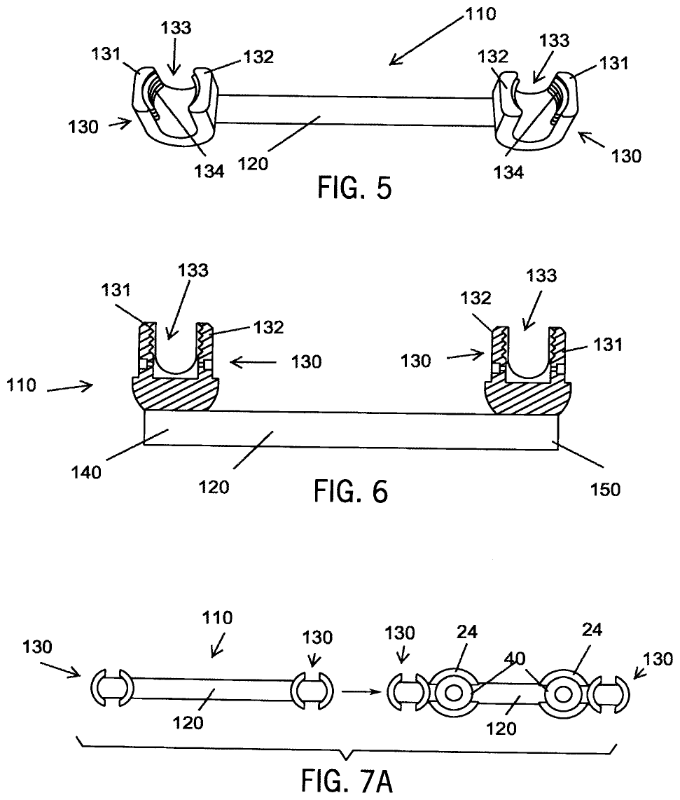

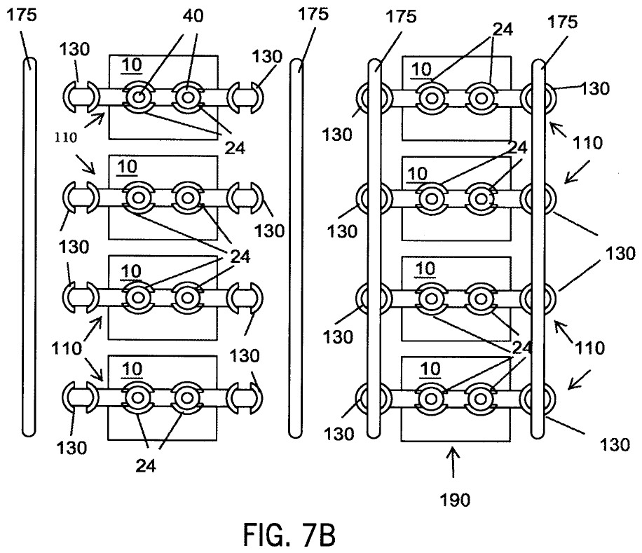

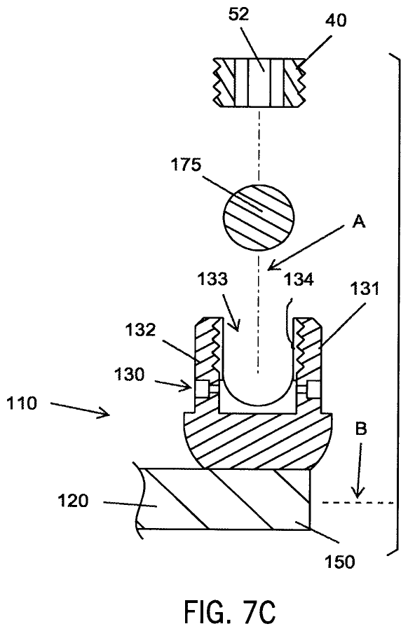

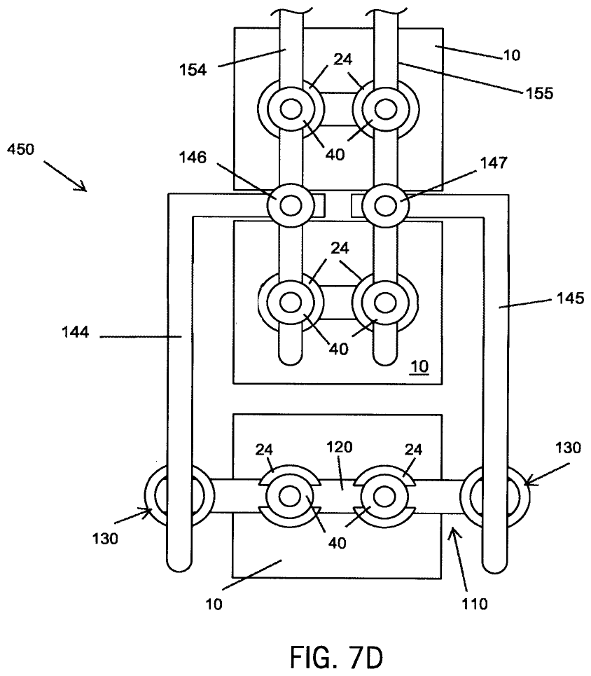

FIGS. 5 and 6 show one example embodiment of a connector 110 suitable for use in a bone surgical apparatus according to the invention. The connector 110 includes a rod 120 having a first end section 140 and a second end section 150 wherein the rod 120 is dimensioned to connect a pair of pedicle screws 24 after the pedicle screws 24 are implanted in a vertebra 10. The rod 120 can be secured to the pedicle screws 24 as described above using the set screw 40. Fasteners 130 are attached to the rod 120 at the first end section 140 and the second end section 150. The fastener 130 comprises a tulip shaped head including spaced apart walls 131, 132 which define a recess 133 for receiving an alignment rod. FIG. 7A shows how the rod 120 of the connector 110 can be assembled to a pair of pedicle screws 24 using set screws 40 as described above with reference to FIGS. 2-4. FIG. 7C shows how an alignment rod 175 can be attached to fasteners 130 of the connector 110 by positioning the alignment rod 175 in the recess 133 between walls 131, 132, and inserting the set screw 40 which can be received in threaded section 134 to thereby restrain alignment rod 175. The set screw 40 can be driven into the head of the fastener 130 by utilizing recess 52. The fasteners 130 can be polyaxial in that a longitudinal axis A of the head of the fastener 130 is movably polyaxial with respect to a longitudinal axis B of the rod 120 before the set screw 40 is received in threaded section 134 to thereby restrain alignment rod 175. See FIG. 7C. Structures for creating a polyaxial head and a head convertible from polyaxial to monoaxial can found, for example, in PCT Patent Application Publication No. WO 2017/035186. Connector 110 can be formed from a metallic material, such as titanium, cobalt chrome or stainless steel.

Turning to FIG. 7B, there is shown is a posterior view showing assembly of an embodiment of a bone surgical apparatus 190 according to the invention. Connectors 110 are assembled to pairs of pedicle screws 24 in adjacent vertebrae 10 using set screws 40 as described above with reference to FIGS. 2-4 as shown on the left side of FIG. 7B. The alignment rods 175 are then assembled to the fasteners 130 using set screws 40 as described above with reference to FIG. 7C as shown on the right side of FIG. 7B. In methods of the invention, because the two pedicle screws provide two points of fixation, the vertebrae turns rather than the pedicle screw within the pedicle when rotational forces are applied. Rotation and compression is possible because the rod 120 is stabilized in the pair of pedicle screws 24 inserted into the pedicles of the same vertebra 10. The rod 120 can be 5.5 millimeters in diameter to fit in standard pedicle screw fastener heads. A percutaneous technique is possible using the pedicle screws 24, the connectors 110, and the alignment rods 175.

Turning to FIG. 7D, there is shown is a posterior view showing assembly of an embodiment of a bone surgical apparatus 450 according to the invention for use in extension of fusion. The top two vertebrae 10 are fused using fusion rods 154, 155 assembled in pairs of pedicle screws 24 in adjacent vertebrae 10 using set screws 40 as described above with reference to FIGS. 2-4. Connector 110 is assembled to pairs of pedicle screws 24 in lower vertebra 10 using set screws 40 as described above with reference to FIGS. 2-4. Then, L-shaped angled rods 144, 145 are assembled to fusion rods 154, 155 using fasteners 146, 147.

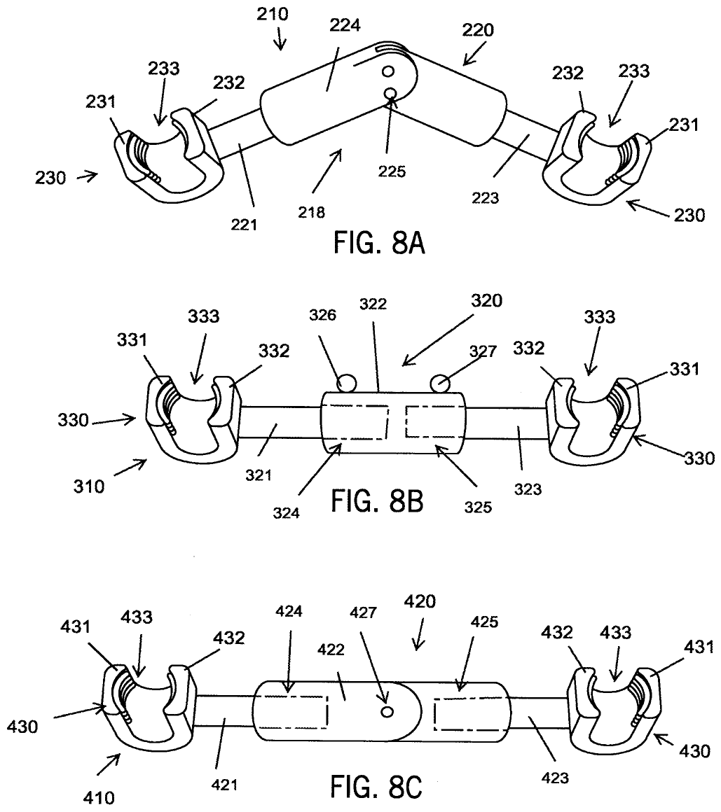

Referring now to FIG. 8A, there is shown a perspective view of an embodiment of an IVIP rod connector 210 suitable for use in a bone surgical apparatus according to the invention. The connector 210 includes a rod assembly 220 having a first end section 221 and a second end section 223 attached to a hinge 224 wherein the rod sections 221, 223 are dimensioned to connect a pair of pedicle screws 24 after the pedicle screws 24 are implanted in a vertebra 10. The rod sections 221, 223 can be secured to the pedicle screws 24 as described above using set screws 40. Fasteners 230 are attached to the rod sections 221, 223. Each fastener 230 comprises a head including spaced apart walls 231, 232 which defining a recess 233 for receiving an alignment rod. FIG. 7C shows how a rod can be attached to fasteners 230 of the connector 210. The fasteners 230 can be polyaxial in that a longitudinal axis A of the head of the fastener 230 is movably polyaxial with respect to a longitudinal axis B of the rod 220 before the set screw 40 is received in a threaded section to thereby restrain alignment rod. See FIG. 7C. Connector 210 can be formed from a metallic material, such as titanium, cobalt chrome or stainless steel.

In the connector 210, the hinge 224 can be freely moving or lockable, The hinge 224 can be locked in place by a set screw 225. Alternatively, a hex nut can be turned to lock separate parts of the hinge together. A captured screw can also be used to lock separate parts of the hinge together.

Turning now to FIG. 8B, there is shown a perspective view of an embodiment of an IVIP rod connector 310 suitable for use in a bone surgical apparatus according to the invention. The connector 310 includes a rod assembly 320 having a first end section 321 and a second end section 323 attached to a sleeve 322 wherein the rod sections 321, 323 are dimensioned to connect a pair of pedicle screws 24 after the pedicle screws 24 are implanted in a vertebra 10. The rod end sections 321, 323 can be secured to the pedicle screws 24 as described above using set screws 40. The length of the rod assembly 320 can be varied by sliding the first end section 321 in a recess 324 of the sleeve 322 and securing the first end section 321 in the recess 324 by a set screw 326, and sliding the second end section 323 in a recess 325 of the sleeve 322 and securing the second end section 323 in the recess 325 by a set screw 327. Fasteners 330 are attached to the rod sections 321, 323. Each fastener 330 comprises a head including spaced apart walls 331, 332 which defining a recess 333 for receiving an alignment rod. FIG. 7C shows how a rod can be attached to fasteners 330 of the connector 310. The fasteners 330 can be polyaxial in that a longitudinal axis A of the head of the fastener 330 is movably polyaxial with respect to a longitudinal axis B of the rod 320 before the set screw 40 is received in a threaded section to thereby restrain alignment rod. See FIG. 7C. Connector 310 can be formed from a metallic material, such as titanium, cobalt chrome or stainless steel.

Referring now to FIG. 8C, there is shown a perspective view of an embodiment of an IVIP rod connector 410 suitable for use in a bone surgical apparatus according to the invention. The connector 410 includes a rod assembly 420 having a first end section 421 and a second end section 423 attached to a hinged sleeve 422 wherein the rod sections 421, 423 are dimensioned to connect a pair of pedicle screws 24 after the pedicle screws 24 are implanted in a vertebra 10. The rod end sections 421, 423 can be secured to the pedicle screws 24 as described above using set screws 40. The length of the rod assembly 420 can be varied by sliding the first end section 421 in a recess 424 of the sleeve 422 and securing the first end section 421 in the recess 424 by a set screw, and sliding the second end section 423 in a recess 425 of the sleeve 422 and securing the second end section 423 in the recess 425 by a set screw. Fasteners 430 are attached to the rod sections 421, 423. Each fastener 430 comprises a head including spaced apart walls 431, 432 which defining a recess 433 for receiving an alignment rod. FIG. 7C shows how a rod can be attached to fasteners 430 of the connector 410. The fasteners 430 can be polyaxial in that a longitudinal axis A of the head of the fastener 430 is movably polyaxial with respect to a longitudinal axis B of the rod 420 before the set screw 40 is received in a threaded section to thereby restrain alignment rod. See FIG. 7C. Connector 410 can be formed from a metallic material, such as titanium, cobalt chrome or stainless steel.

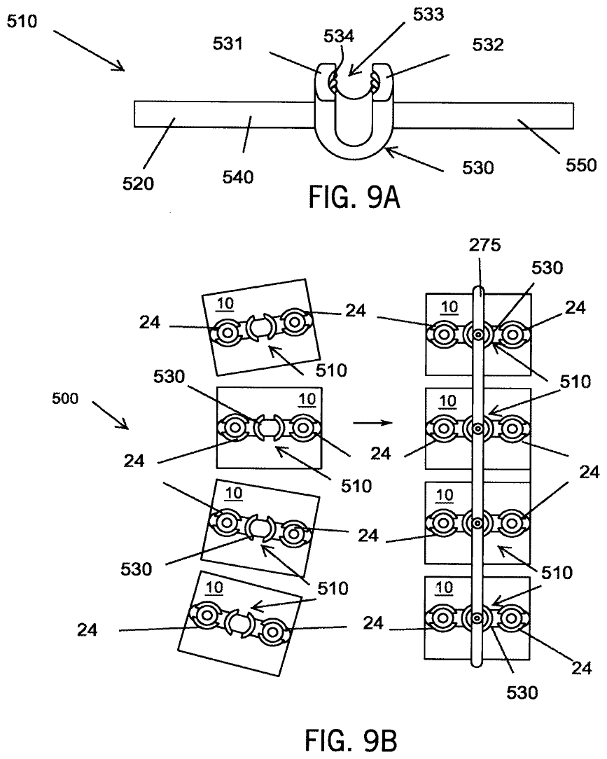

Turning now to FIG. 9A, there is shown a perspective view of an embodiment of an IVIP rod connector 510 suitable for use in a bone surgical apparatus according to the invention. The connector 510 includes a rod 520 having a first end section 540 and a second end section 550 wherein the rod 520 is dimensioned to connect a pair of pedicle screws 24 after the pedicle screws 24 are implanted in a vertebra 10. The rod 520 can be secured to the pedicle screws 24 as described above using the set screw 40. A fastener 530 is attached to the rod 520 between the first end section 540 and the second end section 550. The fastener 530 comprises a head including spaced apart walls 531, 532 which defining a recess 533 for receiving an alignment rod. FIG. 7A shows how the rod 520 of the connector 510 can be assembled to a pair of pedicle screws 24 using set screws 40 as described above with reference to FIGS. 2-4. FIG. 7C shows how an alignment rod can be attached to fastener 530 of the connector 510 by positioning the alignment rod in the recess 533 between walls 531, 532, and inserting the set screw 40 which can be received in threaded section 534 to thereby restrain alignment rod. The fastener 530 can be polyaxial in that a longitudinal axis A of the head of the fastener 530 is polyaxial with respect to a longitudinal axis B of the rod 520 before the set screw 40 is received in threaded section 534 to thereby restrain a rod. See FIG. 7C. Connector 510 can be formed from a metallic material, such as titanium, cobalt chrome or stainless steel.

Referring now to FIG. 9B, there is an assembly of an embodiment of a bone surgical apparatus 500 according to the invention that can be used for fusion. Connectors 510 are assembled to pairs of pedicle screws 24 in adjacent vertebrae 10 using set screws 40 as described above with reference to FIGS. 2-4 as shown on the left side of FIG. 9B. An alignment rod 275 is then assembled to the fasteners 230 using set screws 40 as described above with reference to FIG. 7C as shown on the right side of FIG. 9B.

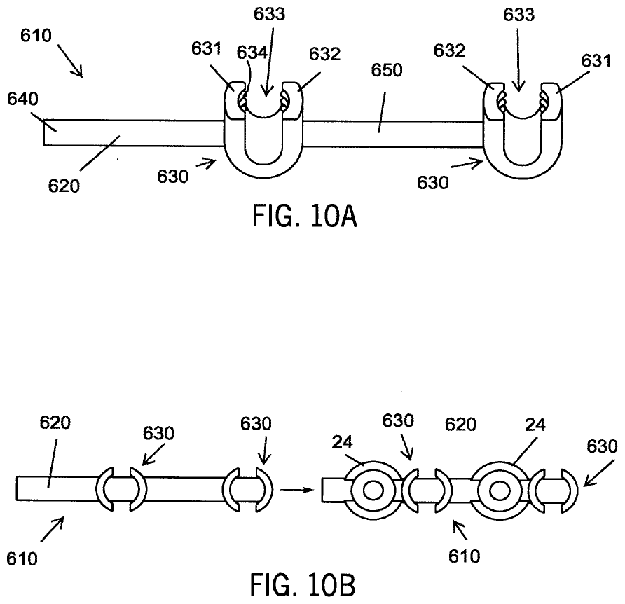

FIG. 10A is a perspective view of another embodiment of an IVIP rod connector 610 suitable for use in a bone surgical apparatus according to the invention. The connector 610 includes a rod 620 having a first end section 640 and a second end section 650 wherein the rod 620 is dimensioned to connect a pair of pedicle screws 24 after the pedicle screws 24 are implanted in a vertebra 10. The rod 620 can be secured to the pedicle screws 24 as described above using the set screw 40. Fasteners 630 are attached to the rod 620 at the second end section 650 and between the first end section 640 and the second end section 650. The fastener 630 comprises a head including spaced apart walls 631, 632 which defining a recess 633 for receiving an alignment rod. FIG. 7A shows how the rod 620 of the connector 610 can be assembled to a pair of pedicle screws 24 using set screws 40 as described above with reference to FIGS. 2-4. FIG. 7C shows how an alignment rod can be attached to fasteners 630 of the connector 610 by positioning the alignment rod in the recess 633 between walls 631, 632, and inserting the set screw 40 which can be received in the threaded section 634 to thereby restrain alignment rod. The fasteners 630 can be polyaxial in that a longitudinal axis A of the head of the fastener 630 is movably polyaxial with respect to a longitudinal axis B of the rod 620 before the set screw 40 is received in threaded section 634 to thereby restrain a rod. Connector 610 can be formed from a metallic material, such as titanium, cobalt chrome or stainless steel. FIG. 10B is a posterior view of the rod 610 connected to a pair of pedicle screws 24.

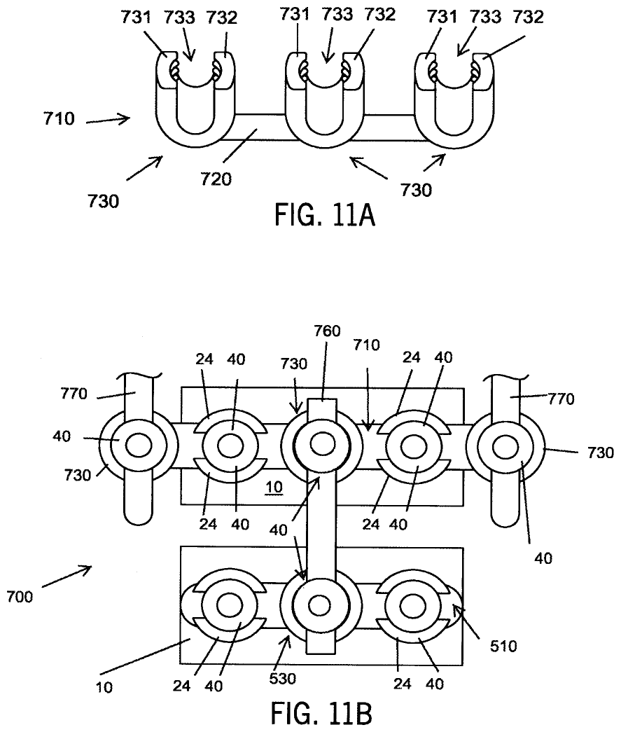

FIG. 11A is a perspective view of another embodiment of an IVIP rod connector 710 suitable for use in a bone surgical apparatus according to the invention. The connector 710 includes a rod 720 dimensioned to connect a pair of pedicle screws 24 after the pedicle screws 24 are implanted in a vertebra 10. The rod 720 can be secured to the pedicle screws 24 as described above using the set screw 40. Fasteners 730 are attached to the rod 720 at the first end section and between the first end section and the second end section, and at the second end section. Each fastener 730 comprises a head including spaced apart walls 731, 732 which define a recess 733 for receiving an alignment rod. FIG. 7A shows how the rod 720 of the connector 710 can be assembled to a pair of pedicle screws 24 using set screws 40 as described above with reference to FIGS. 2-4. FIG. 7C shows how an alignment rod can be attached to fasteners 730 of the connector 710 by positioning the alignment rod in the recess 733 between walls 731, 732, and inserting the set screw 40 which can be received in a threaded section to thereby restrain alignment rod. The fasteners 730 can be polyaxial in that a longitudinal axis A of the head of the fastener 730 is movably polyaxial with respect to a longitudinal axis B of the rod 720 before the set screw 40 is received in threaded section to thereby restrain a rod. Connector 710 can be formed from a metallic material, such as titanium, cobalt chrome or stainless steel.

Referring now to FIG. 11B, there is an assembly of an embodiment of a bone surgical apparatus 700 according to the invention. Connector 510 is assembled to pairs of pedicle screws 24 in lower vertebra 10 using set screws 40 as described above with reference to FIGS. 2-4. Connector 710 is assembled to pairs of pedicle screws 24 in upper vertebra 10 using set screws 40 as described above with reference to FIGS. 2-4. An alignment rod 760 is then assembled to the center fastener 530 of connector 510 and the center fastener 730 of connector 710 using set screws 40 as described above with reference to FIG. 7C. Fusion rods 770 are connected the lateral fasteners 730 of the connector 710.

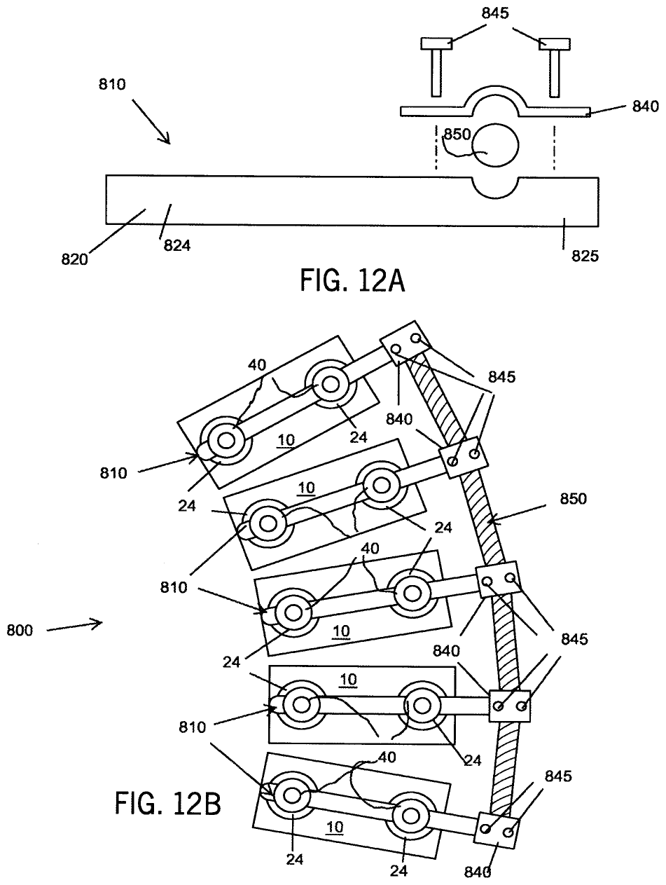

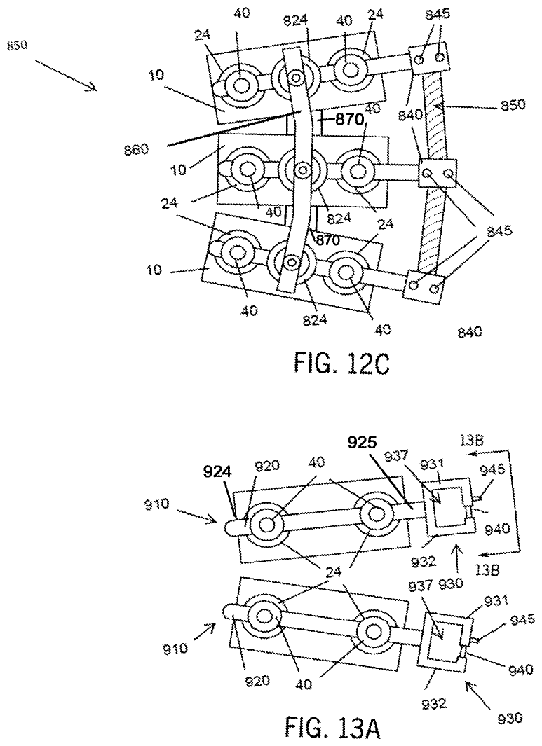

Another embodiment of the invention is particularly advantageous for non-fusion scoliosis surgery. In this embodiment of the invention, there is a clamp at the end of the rod. These rods can be placed such that the clamps are along the convexity of the curve and a band is placed in the clamps such that the convexity of the curve is prevented from lengthening while the concavity of the curve continues to lengthen, resulting in a decrease of the curve. Turning to FIG. 12A, there shown a rod connector 810 suitable for use in a bone surgical apparatus according to the invention. The connector 810 includes a rod 820 having a first end section 824 and a second end section 825 wherein the rod 820 is dimensioned to connect a pair of pedicle screws 24 after the pedicle screws 24 are implanted in a vertebra 10. The rod 820 can be secured to the pedicle screws 24 as described above using the set screw 40. A fastening clamp 840 is attached to the rod 820 at the second end section 825 using fastener screws 845. The clamp 830 receives and restrains a band 850 by tightening the screws 845. Connector 810 can be formed from a metallic material, such as titanium, cobalt chrome or stainless steel. The band 850 can comprise a flexible polymeric material such as polyester.

Referring now to FIG. 12B, there is an assembly of an embodiment of a bone surgical apparatus 800 according to the invention for use in non-fusion scoliosis surgery. Connectors 810 are assembled to pairs of pedicle screws 24 in adjacent vertebrae 10 using set screws 40 as described above with reference to FIGS. 2-4. Band 850 is then assembled to the rods 820 using clamps 840 as shown in FIG. 12A.

One potential problem with posterior tethering is that the spine can go into extension as posterior growth is potentially reduced more than anterior growth. To limit this effect, one can use a rod 820 with a clamp in the middle as well as a clamp 840 along the convexity of the curve as shown in FIG. 12C for use in non-fusion scoliosis surgery. The outer band 850 is placed in the clamps 840 along the convexity of the curve so that the growth along the convexity is tethered. A central band 860 is placed in the middle clamps that are between the pedicle screws 24 along with rigid sheathes 870 that goes over the band 860, between the clamps. These sheaths 870 resist extension of the spine by preventing shortening along the posterior elements of the spine. The transverse cross-sections of the band 850 and the central band 860 can be various shapes, such as circular, oval, polygonal, square, and rectangular.

The present invention also provides for Direct Vertebral ReAlignment (DVRA) using an IVIP rod with a latch for posterior short fusion and non-fusion treatment of scoliosis. Because the two pedicle screws provide two points of fixation in the vertebra, IVIP rods can convert compression force to rotational force that alters the vertebral tilt. IVIP rods can serve as levers such that less force is needed when the force is applied lateral to the pedicle than at the pedicle. The advantages of DVRA include (1) shorter fusions and shorter operative time and blood loss; (2) more anatomic correction; (3) DVRA can also be used in congenital scoliosis instead of growing rods; and (4) the concept is to use the posterior approach but use the concepts for the anterior approach without the risks of the anterior surgery.

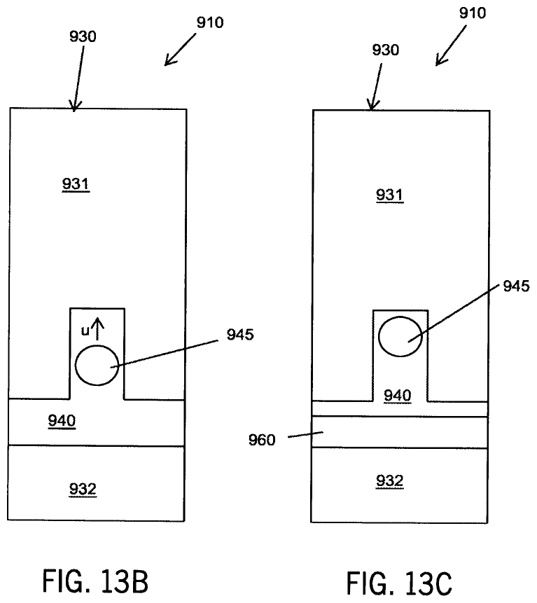

Looking at FIGS. 13A, 13B, and 13C, a connector 910 includes a rod 920 having a first end section 925 and a second end section 924 wherein the rod 920 is dimensioned to connect a pair of pedicle screws 24. A latch 930 is attached to the first end section 925 of the rod 920 wherein the latch 930 comprises a first wall 931 and a second wall 932 defining an interior region 937 for receiving a portion of a loop. The first wall 931 has a first terminal end, and the second wall 932 has a second terminal end. The first terminal end and the second terminal end are positioned to define a space 960 between the first terminal end and the second terminal end. The latch includes a movable barrier 940 with a pin 945 for opening and closing the space 960 between the first terminal end and the second terminal end. A portion of the loop is received in the interior region 937 when the space 960 is open, and the portion of the loop is retained in the interior region 937 when the space 960 is closed. The rod 920 can have a hinge with variable angle and/or a telescoping rod making the length adjustable as in FIGS. 8A-8C. Connector 910 can be formed from a metallic material, such as titanium, cobalt chrome or stainless steel.

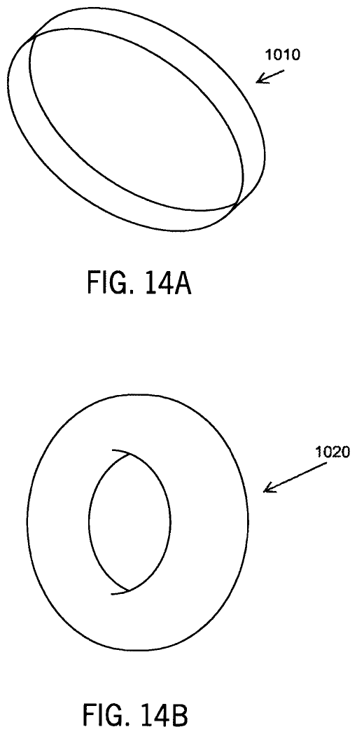

Looking at FIG. 14A, the loop 1010 may be formed from a band of material. Alternatively, the loop is formed from a tubular material (see FIG. 14B). The loop 1010 or loop 1020 can be slid into the interior region 937 through space 960 controlled by the barrier 940. The loop 1010 can comprise a polyethylene or polyester band, similar to that used for spinal fusion. In one form, the loop 1010 can be made of polyester fiber type of non-absorbent material. To make it stronger, loop 1020 is a hollow tube like a float tube.

The latch does not need to have a space providing an opening. One option is to have a tensioning device similar to bands used for spinal fusion. The anchor would be placed on one wall of the latch and a band would go around the limb of the adjacent latch. Another option is to have a way of fusing or suturing or tying the band so that the band can be placed in the adjacent latch without the need for an opening.

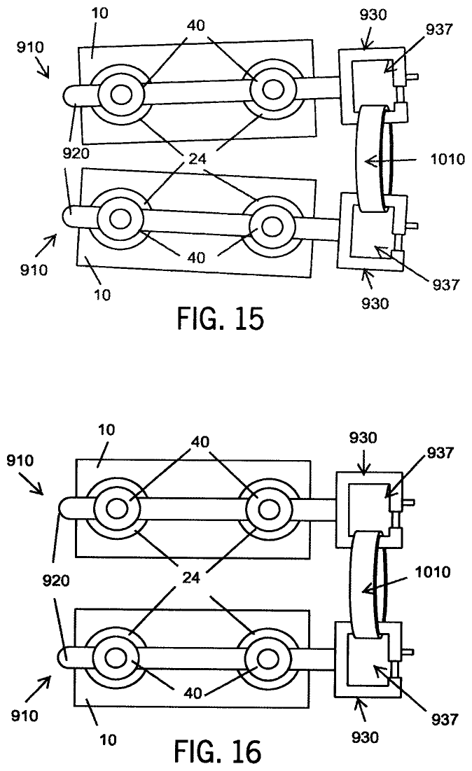

Referring now to FIG. 15, connectors 910 are assembled to pairs of pedicle screws 24 in adjacent vertebrae 10 using set screws 40 as described above with reference to FIGS. 2-4. Then the loop 1010 can be first slid into the interior region 937 of the inferior latch 930, and then the rods 920 are compressed so that the loop 1010 can then be slid into the interior region 937 of the superior latch 930. The loop 1010 placed into the latches 930 can tether the convexity of the curve using the lever advantage of the rods 920. The loops 1010 can have different lengths so that one can compress the convexity a desired amount and distract the concavity; the latter would stimulate the growth along the concavity.

With growth, the convexity is tethered and its growth limited. The concavity will continue to grow, accelerated secondary to distraction, to correct the vertebral tilt between two vertebrae; as the angle between the two vertebrae is corrected at each level, the scoliosis is also corrected. Compare FIG. 15 (right after connecting the loops 1010) to FIG. 16 after correction of the vertebral tilt.

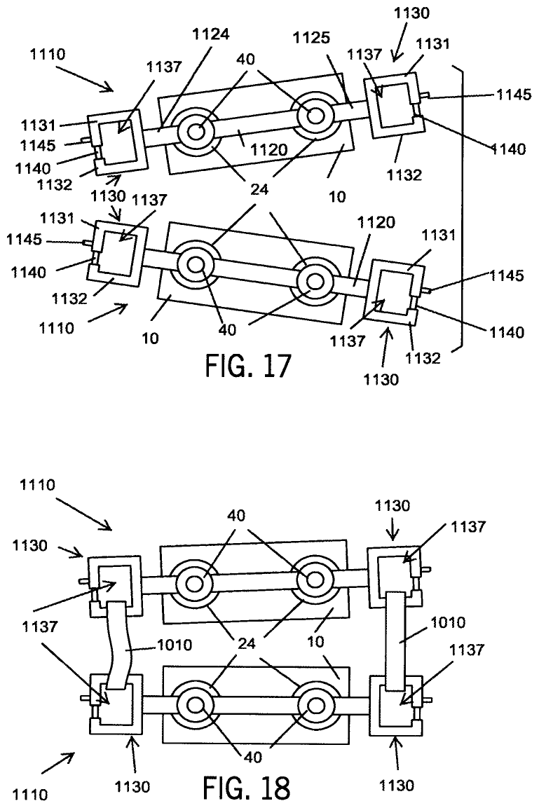

One of the challenges of tethering has been the risk for overcorrection as the convexity of the spine is tethered. A rod with a latch on both ends can be used to prevent overcorrection. Looking at FIG. 17, a connector 1110 includes a rod 1120 having a first end section 1124 and a second end section 1125 wherein the rod 1120 is dimensioned to connect a pair of pedicle screws 24. A latch 1130 is attached to the first end section 1124 and the second end section 1125 of the rod 1120 wherein each latch 1130 comprises a first wall 1131 and a second wall 1132 defining an interior region 1137 for receiving a portion of a loop. The first wall 1131 has a first terminal end, and the second wall 1132 has a second terminal end. The first terminal end and the second terminal end are positioned to define a space (like 960 in FIG. 13C) between the first terminal end and the second terminal end. The latch includes a movable barrier 1140 with a pin 1145 for opening and closing the space between the first terminal end and the second terminal end. A portion of the loop is received in the interior region 1137 when the space is open, and the portion of the loop is retained in the interior region 1137 when the space is closed. The rod 1120 can have a hinge with variable angle and/or a telescoping rod making the length adjustable as in FIGS. 8A-8C. Connector 1110 can be formed from a metallic material, such as titanium, cobalt chrome or stainless steel.

Turning to FIG. 18, after assembling rods 1120 of the connectors 1110 to pairs of pedicle screws 24 in adjacent vertebrae 10 using set screws 40 as described above with reference to FIGS. 2-4, loops 1010 of equal lengths can be placed on each side such that on the convex side of the spine (see, e.g., 14 in FIG. 1), the loop 1010 would act as a tether. On the concave side of the spine (see, e.g., 18 in FIG. 1), the loop 1010 would sit in the latches 1130 without any tension.

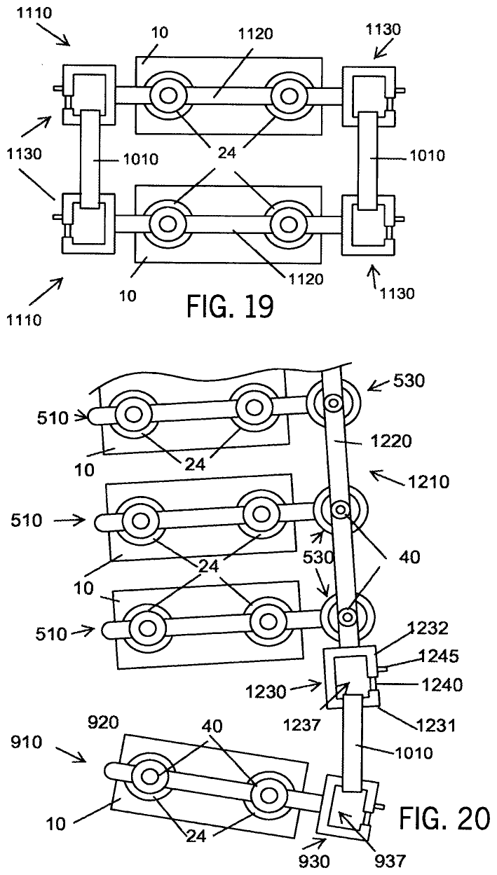

As the concavity grows, the loop 1010 on the left (previous concave side would become taut). As this loop 1010 is the same length as the loop 1010 on the right side, when both sides are at equal length, the tension in the loops 1010 should also be equal, preventing growth on the left side and overcorrection. See FIG. 19.

In addition, in patients with kyphosis, if instrumentation is done with a rod with two ring latches and bands on each side with equal tension as shown in FIG. 19, this would result in tethering of the posterior spine, causing relative more growth along the anterior of the spine. The greater growth along the anterior spine would result in correction of kyphosis. Therefore, the rods with two ring latches can also be used in the correction of kyphosis of a growing child, saving the patient from a fusion surgery.

In some clinical situations, the treatment of the spine involves a combination of fusion linked to tethering. This approach allows for limited fusion in spines with little to no growing potential. The present invention also allows for fusion of the primary curve and tethering of the secondary curve. One method to accomplish this is to place the latch in the side limb, rather than at the end. The rod portion would be used for fusion and a band can be placed in the rings for tethering. Turning to FIG. 20, there is shown an embodiment of a bone surgical apparatus according to the invention. A connector 1210 includes a rod 1220 having a first end section and a second end section wherein the rod 1220 is dimensioned to connect a pair of pedicle screws 24. A latch 1230 is attached to the first end section of the rod 1220 wherein the latch 1230 comprises a first wall 1231 and a second wall 1232 defining an interior region 1237 for receiving a portion of a loop. The first terminal end and the second terminal end are positioned to define a space (like 960 in FIG. 13C) between the first terminal end and the second terminal end. The latch 1230 includes a movable barrier 1240 with a pin 1245 for opening and closing the space between the first terminal end and the second terminal end. A portion of the loop 1010 is received in the interior region 1237 when the space is open, and the portion of the loop is retained in the interior region 1237 when the space is closed.

Still looking at FIG. 20, connectors 510 are assembled to pairs of pedicle screws 24 in adjacent vertebrae 10 using set screws 40 as described above with reference to FIGS. 2-4. Rod 1220 is then assembled to the fasteners 530 using set screws 40 as described above with reference to FIG. 7C. Connector 910 is assembled to pairs of pedicle screws 24 in the lower vertebrae 10 using set screws 40 as described above with reference to FIGS. 2-4. Then the loop 1010 can be first slid into the interior region 937 of the latch 930, and then the rods 520, 920 are compressed so that the loop 1010 can then be slid into the interior region 1237 of the superior latch 1230. The loop 1010 placed into the latches 930, 1230 can tether the convexity of the curve using the lever advantage of the rods 520, 920.

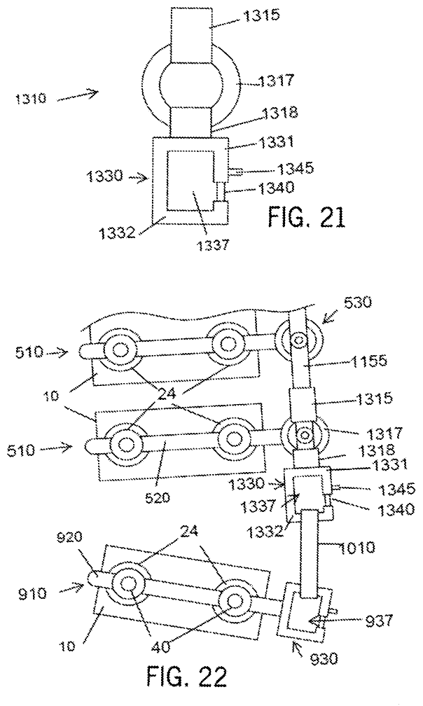

Another option is a latch connector device that includes multiple parts. A first part is a latch attached to a central section wherein the latch comprises a first wall and a second wall defining an interior region for receiving a portion of a loop. A second part is a central section that fits around a fastener and the rod in the fastener. A third part is a rod adapter sits around the rod and can be fastened to the rod, for example, by an interference screw. Referring to FIG. 21, there is shown an embodiment of a latch connector according to the invention. The latch connector 1310 comprising a rod adapter 1315 dimensioned to attach to a fusion rod, a central section 1317 attached to the rod adapter 1315 wherein the central section is dimensioned to attach to a head of a pedicle screw, and a latch 1330 attached to the central section 1317 by a fastener 1318 wherein the latch 1330 comprises a first wall 1331 and a second wall 1332 defining an interior region 1337 for receiving a portion of a loop.

Looking at FIG. 22, connectors 510 are assembled to pairs of pedicle screws 24 in adjacent vertebrae 10 using set screws 40 as described above with reference to FIGS. 2-4. A fusion rod 1155 is then assembled to the fasteners 530 using set screws 40 as described above with reference to FIG. 7C. Connector 910 is assembled to pairs of pedicle screws 24 in the lower vertebrae 10 using set screws 40 as described above with reference to FIGS. 2-4. The central section 1317 of the latch connector 1310 is placed around the most inferior fastener 530 and rod 1155 and fastened to the fusion rod 1155. Then tethering can be done by placing a band between the two adjacent latches 930, 1330. The loop 1010 can be first slid into the interior region 937 of the latch 930, and then the rods 520, 920 are compressed so that the loop 1010 can then be slid into the interior region 1337 of the superior latch 1330.

The loop 1010 placed into the latches 930, 1330 can tether the convexity of the curve using the lever advantage of the rods 520, 920.

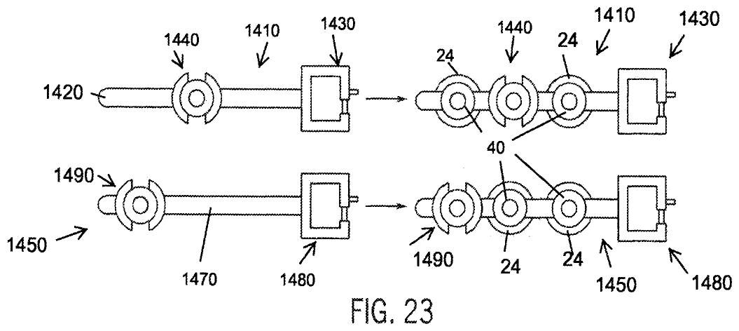

Another variation of the invention is a rod with a latch and a polyaxial fastener. The fastener could be at the end of the rod or the middle of the rod. The rod portion can have a hinge with variable angle and/or a telescoping rod making the length adjustable as in FIGS. 8A-8C. Referring to FIG. 23, a connector 1410 includes a rod 1420 having a latch 1430 (similar to latch 930) attached to an end section of the rod 1420. Fastener 1440 is attached to the rod 1420 between the first end section and the second end section. The fastener 1440 is similar to fastener 130 described above. Connector 1410 can be formed from a metallic material, such as titanium, cobalt chrome or stainless steel. Connector 1410 can be assembled to pairs of pedicle screws 24 in adjacent vertebrae 10 using set screws 40 as described above with reference to FIGS. 2-4. This leaves fastener 1440 available for connection to a fusion rod or an alignment rod as described above with reference to FIG. 7C.

Still referring to FIG. 23, another connector 1450 includes a rod 1470 having a latch 1480 (similar to latch 930) attached to an end section of the rod 1470. Fastener 1490 is attached to the rod 1420 near the first end section. The fastener 1490 is similar to fastener 130 described above. Connector 1450 can be formed from a metallic material, such as titanium, cobalt chrome or stainless steel. Connector 1450 can be assembled to pairs of pedicle screws 24 in adjacent vertebrae 10 using set screws 40 as described above with reference to FIGS. 2-4. This leaves fastener 1490 available for connection to a fusion rod or an alignment rod as described above with reference to FIG. 7C.

Thus, there are at least three applications of IVIP rods of the invention: (1) fusion for pediatric and young adult spinal deformity; (2) special situations such as extension of fusion; and (3) non-fusion scoliosis surgery, often described as “tethering”, using a posterior approach to the spine (tethering for scoliosis has been almost exclusively done anteriorly).

The advantage of the rods of the invention is that it also acts as a lever. For example, the center of rotation between adjacent vertebrae is at the center of the disc. Using current techniques, the forces are applied at the pedicles.
fᵢ=input force, compression
d₁=distance from fᵢ to center of rotation
fₒ=output force at the edge of vertebra
d₂=distance from fₒ to center of rotation
The equation to describe this lever is: fᵢ×d₁=fₒ×d₂.
Since d₁ is less than d₂, the output force fₒ is a fraction of fᵢ.
   But with the IVIP rods, d₁ is greater than d₂
Again, fᵢ×d₁=fₒ×d₂
Since d₁ is 2 to 3 times larger than d₂, fₒ is 2-3 times greater than fᵢ when rods are used.
Therefore, rods of the invention result in mechanical advantage such that for a given fₒ, the input force i would be ½ to ⅓ of fₒ.
Without rods of the invention, the input force would be 2-3 times the fₒ.
Defining fₒ as force necessary for the correction of the angulation between the vertebrae, the rods of the invention afford a four to nine fold mechanical advantage, i.e., much less force is needed to achieve the same correction. By having the length of the rods adjustable, the mechanical advantage can be increased as needed within the limits of local anatomy.
   Having fasteners on both sides of the rods of the invention allow the surgeon to use the fasteners of the convex side to apply force to correct the deformity and to use the fasteners on the concavity to lock in the correction by placing a rod into the tulip shaped heads and stabilizing the rod using set screws.

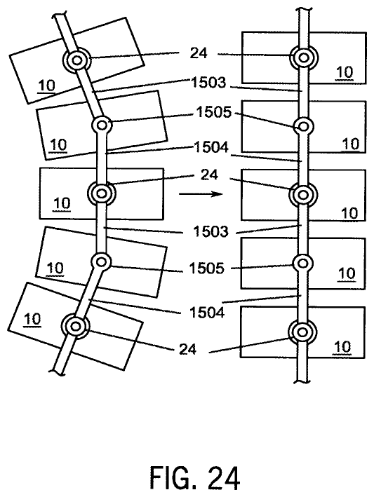

FIG. 24 is a posterior view showing another embodiment of a bone surgical apparatus for manipulating a curvature of a spine. The apparatus incudes a pair of adjustment mechanisms including a first rod section 1503 and a second rod section 1504 connected by a hinge 1505. As shown in the left hand side of FIG. 24, the first rod sections 1503 and the second rod sections 1504 are assembled to pairs of pedicle screws 24 in vertebrae 10 using set screws 40 as described above with reference to FIGS. 2-4. Pressure is applied to straighten the adjustment mechanisms to the position shown on the right hand side of FIG. 24, and the adjustment mechanisms can be held in the position shown on the right hand side of FIG. 24 using locking of the hinges or an external fixator. In another embodiment, the hinge 1505 can include a directional ratchet as described in U.S. Pat. No. 10,463,400, which is incorporated herein by reference as if set forth in its entirety for all purposes. The ratchet can control motion in both of a first rotational direction and an opposite second rotational direction of the hinge 1505.

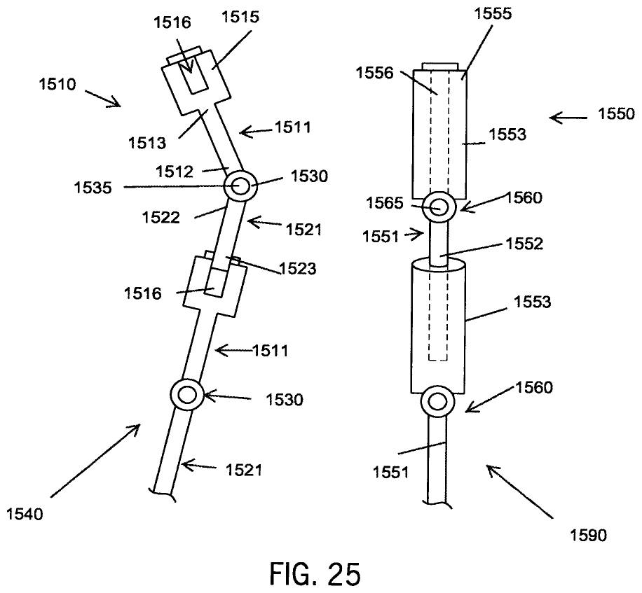

FIG. 25 is a posterior view showing embodiments of alternative adjustment mechanisms for a bone surgical apparatus according to the invention. The adjustment mechanism 1510 comprises a first rod section 1511 having an inner end 1512 and an outer end 1513, a second rod section 1521 having an inner end 1522 and an outer end 1523 having an outer surface shape, and a hinge 1530 attached to the inner end 1512 of the first rod section 1511 and to the inner end 1522 of the second rod section 1521. The outer end 1513 of the first rod section 1511 comprises a wall 1515 defining a recess 1516 dimensioned for receiving a rod end section having a perimeter shaped the same as the outer surface shape of the outer end 1523 of the second rod section 1521. A first section of the hinge is attached to the inner end 1512 of the first rod section 1511, a second section of the hinge is attached to the inner end 1522 of the second rod section 1521, and the hinge 1530 includes a locking device for locking the hinge 1530 in one of a plurality of relative positions between the first section of the hinge 1530 and the second section of the hinge 1530. The hinge 1530 can be locked in place by a set screw 1535. Alternatively, a hex nut can be turned to lock separate parts of the hinge together. A captured screw can also be used to lock separate parts of the hinge together. The bone surgical apparatus includes a second adjustment mechanism 1540 identical to the adjustment mechanism 1510.

The first rod section 1511 and the second rod section 1521 of the adjustment mechanism 1510 are assembled to pairs of pedicle screws 24 in vertebrae 10 using set screws 40 in the general arrangement shown in the embodiment of FIG. 24. The outer end 1523 of the second rod section 1521 can then be inserted in the recess 1516 of the second adjustment mechanism 1540 and secured with a set screw or an interference fit, and the second rod section 1521 of the second adjustment mechanism 1540 is assembled to a pedicle screw 24. Pressure is applied to straighten the adjustment mechanisms 1510, 1540 to a position shown on the right hand side of FIG. 24, and the adjustment mechanisms can be held in the position shown on the right hand side of FIG. 24 using locking of the hinges 1530. In another embodiment, the hinge 1530 can include a directional ratchet as described in U.S. Pat. No. 10,463,400. The ratchet can control motion in both of a first rotational direction and an opposite second rotational direction of the hinge 1530.

In an alternative embodiment in FIG. 25, the adjustment mechanism 1550 comprises a rod section 1551 having an inner end 1552, a sleeve 1553 having a wall 1555 defining a recess 1556 dimensioned for receiving a rod end section having a perimeter shaped the same as the outer surface shape of the inner end 1552 of the rod section 1551, and a hinge 1560 attached to the rod section 1551 and to the sleeve 1553. The hinge 1560 can be locked in place by a set screw 1565. Alternatively, a hex nut can be turned to lock separate parts of the hinge together. A captured screw can also be used to lock separate parts of the hinge together. The bone surgical apparatus includes a second adjustment mechanism 1590 identical to adjustment mechanism 1550. The rod section 1551 of the adjustment mechanism 1550 is assembled to a pedicle screw 24 in vertebrae 10 using a set screw 40 in the general arrangement shown in the embodiment of FIG. 24. The inner end 1552 of the rod section 1551 can then be inserted in the recess 1556 of the second adjustment mechanism 1590 and secured with a set screw or an interference fit, and the rod section 1551 of the second adjustment mechanism 1590 is assembled to a pedicle screw 24. Pressure is applied to straighten the adjustment mechanisms 1550, 1590 to a position shown on the right hand side of FIG. 24, and the adjustment mechanisms can be held in the position shown on the right hand side of FIG. 24 using locking of the hinges 1560.

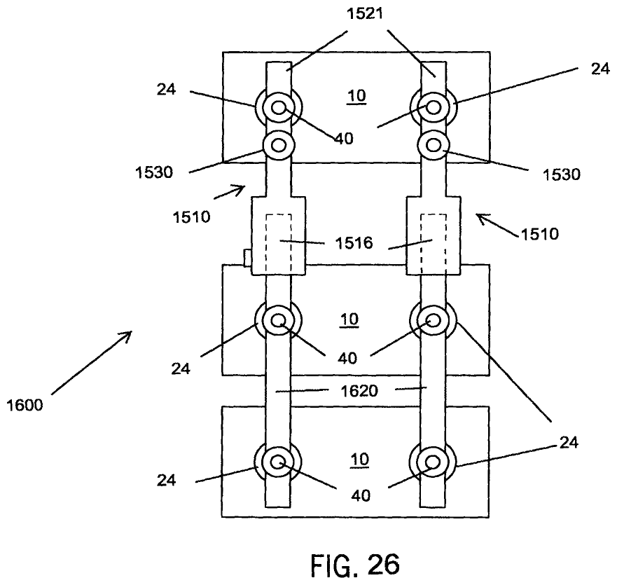

Turning now to FIG. 26, another embodiment of a bone surgical apparatus 1600 according to the invention is shown. The second rod sections 1521 of the adjustment mechanisms 1510 are assembled to pedicle screws 24 in vertebrae 10 using set screws 40. The inner ends 1623 of fusion rods 1620 can then be inserted in the recess 1516 of the adjustment mechanisms 1510 and secured with a set screw or an interference fit. The outer ends 1623 of fusion rods 1620 can then be assembled to pedicle screws 24 in vertebrae 10 using set screws 40. Pressure is applied to straighten the adjustment mechanisms 1510, and the adjustment mechanisms 1510 can be held in place by locking the hinges 1530. In another embodiment, the hinge 1530 can include a directional ratchet as described in U.S. Pat. No. 10,463,400. The ratchet can control motion in both of a first rotational direction and an opposite second rotational direction of the hinge 1530. Also in FIG. 26, one can rotate the apparatus by 90 degrees such that the hinge/ratchet is along the sagittal plane rather than the coronal plan and get correction in the sagittal plane for either kyphosis or lordosis. The advantage of the ratchet is that it allows for correction of the deformity that does not depend wholly on the growth along the concavity of the curve while the convexity of the curve is being tethered by the apparatus.

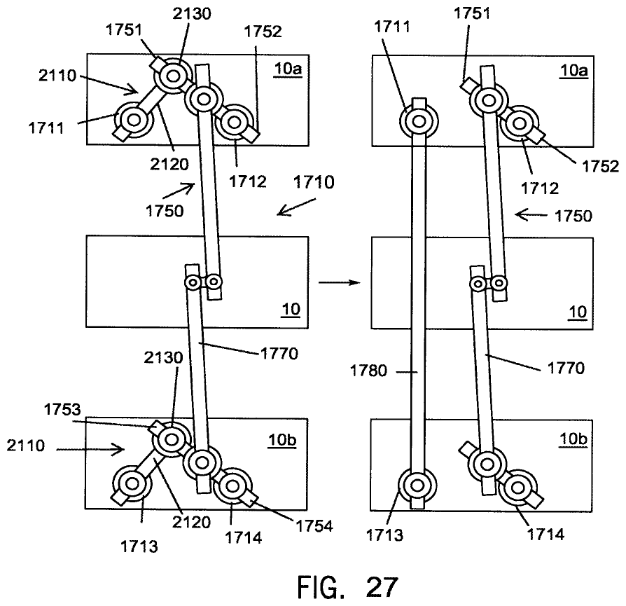

Referring to FIG. 27, there is another embodiment of a bone surgical apparatus 1710 according to the invention. The apparatus 1710 includes a first pedicle screw 1711 and a second pedicle screw 1712 attached to a first vertebra 10a, and a third pedicle screw 1713 and a fourth pedicle screw 1714 attached to a second vertebra 10b. Connectors 2110 include a rod 2120 and a fastener 2130 are attached to the rod 2120 at an end section. The fastener 2130 comprises a head including spaced apart walls which defining a recess for receiving a rod (as in FIG. 7C). The rod 2120 of one connector 2110 is assembled to the first pedicle screw 1711. The rod 2120 of the other connector 2110 is assembled to the third pedicle screw 1713. The fasteners 2130 can be polyaxial in that a longitudinal axis A of the head of the fastener 2130 is movably polyaxial with respect to a longitudinal axis B of the rod 2120 before the set screw 40 is received in threaded section. Connector 2110 can be formed from a metallic material, such as titanium, cobalt chrome or stainless steel.

Still referring to FIG. 27, the apparatus 1710 includes a fixator 1750 having a first end section 1751, a second end section 1752, a third end section 1753, and a fourth end section 1754. The first end section 1751 of the fixator 1750 is attached to the fastener 2130 of one connector 2110, and the second end section 1752 of the fixator 1750 is attached to the second pedicle screw 1712. The third end section 1753 of the fixator 1750 is attached to the fastener 2130 of the other connector 2110, and the fourth end section 1754 of the fixator 1750 is attached to the fourth pedicle screw 1714. The fixator 1750 includes a hinged alignment rod 1770. In a method for manipulating a curvature of a spine of a subject, the bone surgical apparatus 1710 is implanted into a subject as shown in the left hand side of FIG. 27 such that the first pedicle screw 1711 and the second pedicle screw 1712 are attached to the vertebra 10a and the third pedicle screw 1713 and the fourth pedicle screw 1714 are attached to the second vertebra 10b. The fixator 1710 is moved using hinged alignment rod 1770 to achieve a desired alignment of the vertebra 10a and the vertebra 10b. The connectors 2110 are removed from the first pedicle screw 1711 and the third pedicle screw 1713 by removing the set screws; and an alignment rod 1780 is attached to the first pedicle screw 1711 and the third pedicle screw 1713 as shown in the right hand side of FIG. 27. The fixator 1750 can be removed from the third pedicle screw 1713 and the fourth pedicle screw 1714, and a second alignment rod can be attached to the third pedicle screw 1713 and the fourth pedicle screw 1714.

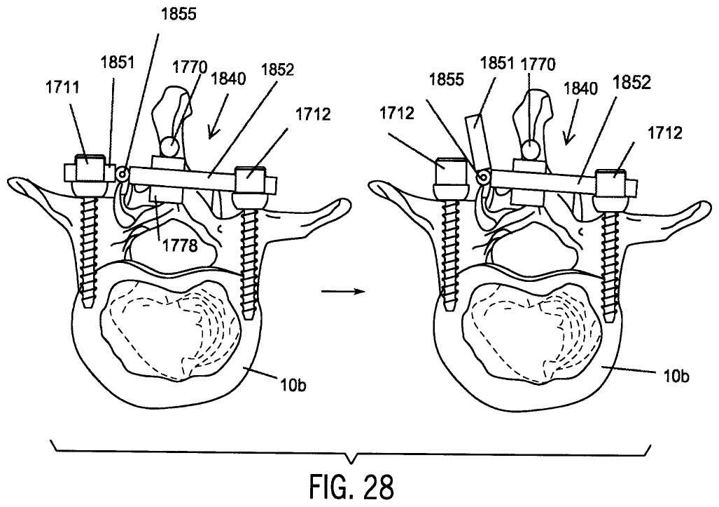

Looking now at FIG. 28, the fixator 1750 of FIG. 27 can have an alternative arrangement for the first end section, the second end section, the third end section, and the fourth end section. Attached to the hinged alignment rod 1770 of the fixator 1750 via clamp 1778 is a hinged rod 1840 having a first end 1851 received in a recess in a head of the first pedicle screw 1711 for attachment to the first pedicle screw 1711, and having a second end 1852 received in a recess in a head of the second pedicle screw 1712 for attachment to the second pedicle screw 1712 as shown in the left hand side of FIG. 28. A second hinged rod identical to hinged rod 1840 is attached to the third pedicle screw 1713 and the fourth pedicle screw 1714. In a method for manipulating a curvature of a spine of a subject, the fixator is moved to achieve a desired alignment of the first vertebra 10a and the second vertebra 10b, and the first end 1851 is removed via movement of hinge 1855 as shown in the right hand side of FIG. 28 and by removing the set screws from the first pedicle screw 1711, and the first end of the second hinged rod is removed via movement of the hinge from the third pedicle screw 1713. An alignment rod 1780 is attached to the first pedicle screw 1711 and the third pedicle screw 1713 as shown in the right hand side of FIG. 27. The fixator can be removed from the third pedicle screw 1713 and the fourth pedicle screw 1714, and a second alignment rod can be attached to the third pedicle screw 1713 and the fourth pedicle screw 1714.

Thus, the present invention provides a bone surgical apparatus and method for manipulating a curvature of a spine.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

What is claimed is:

1. A bone surgical apparatus for manipulating a curvature of a spine, the bone surgical apparatus comprising:
a connector comprising a rod having a first end section and a second end section wherein the rod is dimensioned to connect a pair of pedicle screws, and a fastener attached to the rod wherein the fastener comprises a head including spaced apart walls defining a recess for receiving an alignment rod.

2. The bone surgical apparatus of claim 1 wherein:
the fastener is located at the first end section of the rod.

3. The bone surgical apparatus of claim 1 wherein:
the fastener is located between the first end section and the second end section of the rod.

4. The bone surgical apparatus of claim 1 further comprising:
an additional fastener attached to the rod wherein the additional fastener comprises a head including spaced apart walls defining a recess for receiving an alignment rod.

5. The bone surgical apparatus of claim 4 wherein:
the fastener is located at the first end section of the rod, and
the additional fastener is located at the second end section of the rod.

6. The bone surgical apparatus of claim 1 further comprising:
a plurality of additional fasteners attached to the rod, wherein at least one of the additional fasteners comprises a head including spaced apart walls defining a recess for receiving an alignment rod.

7. The bone surgical apparatus of claim 1 further comprising:
a second fastener attached to the rod wherein the second fastener comprises a head including spaced apart walls defining a recess for receiving an alignment rod, and
a third fastener attached to the rod wherein the third fastener comprises a head including spaced apart walls defining a recess for receiving an alignment rod,
wherein the fastener is located at the first end section of the rod,
wherein the second fastener is located at the second end section of the rod, and
wherein the third fastener is located between the first end section and the second end section of the rod.

8. The bone surgical apparatus of claim 1 wherein:
a longitudinal axis of the head of the fastener is polyaxial with respect to a longitudinal axis of the rod.

9. The bone surgical apparatus of claim 1 further comprising:
a pair of pedicle screws configured to attach to a vertebra and dimensioned to receive the rod of the connector.

10. The bone surgical apparatus of claim 1 further comprising:
a first pair of pedicle screws configured to attach to a first vertebra, the first pair of pedicle screws being attached to the rod of the connector,
a second connector comprising a second rod having a first end section and a second end section wherein the second rod is dimensioned to connect a pair of pedicle screws, and a second fastener attached to the second rod wherein the second fastener comprises a head including spaced apart walls defining a recess for receiving the alignment rod, and
a second pair of pedicle screws configured to attach to a second vertebra, the second pair of pedicle screws being attached to the second rod of the second connector.

11. The bone surgical apparatus of claim 10 further comprising:
an alignment rod connected to the fastener of the rod and the second fastener of the second rod.

12. The bone surgical apparatus of claim 11 wherein:
the fastener is located between the first end section and the second end section of the rod, and
the second fastener is located between the first end section and the second end section of the second rod.

14. The bone surgical apparatus of claim 11 further comprising:
a first additional fastener attached to the first end section of the rod.

15. The bone surgical apparatus of claim 14 further comprising:
a second additional fastener attached to the second end section of the rod.

16. The bone surgical apparatus of claim 15 further comprising:
a second alignment rod connected to the first additional fastener and the second additional fastener, and
a third alignment rod connected to the first additional fastener and the second additional fastener.

17. The bone surgical apparatus of claim 11 wherein:
the fastener is located at the first end section of the rod, and
the second fastener is located at the first end section of the second rod.

18. The bone surgical apparatus of claim 17 further comprising:
a first additional fastener attached to the second end section of the rod,
a second additional fastener attached to the second end section of the second rod, and
a second alignment rod connected to the first additional fastener and the second additional fastener.

19. The bone surgical apparatus of claim 18 wherein:
the first additional fastener comprises a head including spaced apart walls defining a recess for receiving the second alignment rod, and
the second additional fastener comprises a head including spaced apart walls defining a recess for receiving the second alignment rod.

20. The bone surgical apparatus of claim 1 further comprising:
a first pair of pedicle screws configured to attach to a first vertebra, the first pair of pedicle screws being attached to the rod of the connector;
an additional pedicle screw configured to attach to a second vertebra, the additional pedicle screw being connected to a fusion rod; and
an angled rod,
wherein the fastener is located at the first end section of the rod, and
wherein the angled rod is connected to the fastener and the fusion rod.

21. The bone surgical apparatus of claim 1 further comprising:
a first pair of pedicle screws configured to attach to a first vertebra, the first pair of pedicle screws being attached to the rod of the connector;
an additional pedicle screw configured to attach to a second vertebra, the additional pedicle screw being connected to a fusion rod; and
an angled rod,
wherein the fastener is located at the first end section of the rod, and
wherein the angled rod is connected to the fastener and the fusion rod.

22. The bone surgical apparatus of claim 1 wherein:
the first end section of the rod and the second end section of the rod are connected by a hinge.

23. The bone surgical apparatus of claim 22 wherein:
a first section of the hinge is attached to the first end section of the rod,
a second section of the hinge is attached to the second end section of the rod, and
the hinge includes a locking device for locking the hinge in one of a plurality of relative positions between the first section of the hinge and the second section of the hinge.

24. The bone surgical apparatus of claim 1 wherein:
the first end section of the rod and the second end section of the rod are connected by an adjustment mechanism structured to retain the first end section of the rod and the second end section of the rod at any of a plurality of distances between the first end section of the rod and the second end section of the rod thereby providing for variable lengths of the rod.

25. The bone surgical apparatus of claim 24 wherein:
the adjustment mechanism is connected by a hinge to at least one of the first end section of the rod and the second end section of the rod.

26. The bone surgical apparatus of claim 1 further comprising:
a first pedicle screw and a second pedicle screw configured to attach to a first vertebra, the rod of the connector being attached to the first pedicle screw, the fastener being located at the first end section of the rod;
a third pedicle screw and a fourth pedicle screw configured to attach to a second vertebra; and
a fixator having a first end section and a second end section, the first end section of the fixator being attached to the fastener and the second pedicle screw, and the second end section of the fixator being attached to the fourth pedicle screw.

27. The bone surgical apparatus of claim 26 wherein:
the first end section of the fixator comprises an alignment rod,
a first end of the alignment rod is received in the recess of the fastener for attachment to the fastener, and
a second end of the alignment rod is received in a recess in a head of the second pedicle screw for attachment to the second pedicle screw.

28. A bone surgical apparatus for manipulating a curvature of a spine, the bone surgical apparatus comprising:
a connector comprising a rod having a first end section and a second end section wherein the rod is dimensioned to connect a pair of pedicle screws, and a fastener attached to the rod wherein the fastener comprises sections for clamping a band.

29. The bone surgical apparatus of claim 28 wherein:
the fastener is located at the first end section of the rod.

30. The bone surgical apparatus of claim 28 further comprising:
a second fastener attached to the rod wherein the second fastener comprises sections for clamping a second band,
wherein the fastener is located at the first end section of the rod,
wherein the second fastener is located between the first end section and the second end section of the rod.

31. The bone surgical apparatus of claim 28 further comprising:
a first pair of pedicle screws configured to attach to a first vertebra, the first pair of pedicle screws being attached to the rod of the connector,
a second connector comprising a second rod having a first end section and a second end section wherein the second rod is dimensioned to connect a pair of pedicle screws, and a second fastener attached to the second rod, wherein the second fastener comprises a sections for clamping a band;
a second pair of pedicle screws configured to attach to a second vertebra, the second pair of pedicle screws being attached to the second rod of the second connector, and
a band connected to the fastener and the second fastener.

32. The bone surgical apparatus of claim 31 further comprising:
a first additional fastener attached to the rod between the first end section and the second end section of the rod;
a second additional fastener attached to the second rod between the first end section and the second end section of the second rod;
a second band connected to the first additional fastener and the second additional fastener; and
a sheath surrounding the second band.

33. A bone surgical apparatus for manipulating a curvature of a spine, the bone surgical apparatus comprising:
a connector comprising a rod having a first end section and a second end section wherein the rod is dimensioned to connect a pair of pedicle screws, and a latch attached to the first end section of the rod wherein the latch comprises a first wall and a second wall defining an interior region for receiving a portion of a loop.

34. The bone surgical apparatus of claim 32 further comprising:
an additional latch attached to the second end section of the rod wherein the additional latch comprises a first wall and a second wall defining an interior region for receiving a portion of a loop.

35. The bone surgical apparatus of claim 32 wherein:
the first wall has a first terminal end,
the second wall has a second terminal end,
the first terminal end and the second terminal end are positioned to define a space between the first terminal end and the second terminal end,
the latch includes a movable barrier for opening and closing the space between the first terminal end and the second terminal end,
the portion of the loop is received in the interior region when the space is open, and
the portion of the loop is retained in the interior region when the space is closed.

36. The bone surgical apparatus of claim 33 further comprising:
a first pair of pedicle screws configured to attach to a first vertebra, the first pair of pedicle screws being attached to the rod of the connector,
a second connector comprising a second rod having a first end section and a second end section wherein the second rod is dimensioned to connect a pair of pedicle screws, and a second latch attached to the first end section of the second rod wherein the second latch comprises a first wall and a second wall defining an interior region for receiving a portion of the loop,
a second pair of pedicle screws configured to attach to a second vertebra, the second pair of pedicle screws being attached to the second rod of the second connector, and
a loop having a first portion retained in the interior region of the latch and a second portion retained in the interior region of the second latch.

37. The bone surgical apparatus of claim 36 wherein:
the loop is dimensioned such that the loop is tensioned when retained in the interior region of the latch and retained in the interior region of the second latch.

38. The bone surgical apparatus of claim 36 further comprising:
a first additional latch attached to the second end section of the rod, wherein the first additional latch comprises a first wall and a second wall defining an interior region for receiving a portion of a loop;
a second additional latch attached to the second end section of the second rod, wherein the second additional latch comprises a first wall and a second wall defining an interior region for receiving a portion of a loop; and
a second loop having a first portion retained in the interior region of the first additional latch and a second portion retained in the interior region of the second additional latch.

39. The bone surgical apparatus of claim 33 further comprising:
a first pair of pedicle screws configured to attach to a first vertebra, the first pair of pedicle screws being attached to the rod of the connector;
a second connector comprising a second rod having a first end section and a second end section wherein the second rod is dimensioned to connect a pair of pedicle screws, and a fastener attached to the second rod wherein the fastener comprises a head including spaced apart walls defining a recess for receiving a rod;
a second pair of pedicle screws configured to attach to a second vertebra, the second pair of pedicle screws being attached to the second rod of the second connector;
a third connector comprising a third rod having a first end section and a second end section wherein the third rod is dimensioned to connect a pair of pedicle screws, and a second latch attached to the first end section of the third rod wherein the second latch comprises a first wall and a second wall defining an interior region for receiving a portion of the loop, the third rod being attached to the fastener of the second connector; and
a loop having a first portion retained in the interior region of the latch and a second portion retained in the interior region of the second latch.

40. The bone surgical apparatus of claim 36 wherein:
the loop is formed from a band of material.

41. The bone surgical apparatus of claim 36 wherein:
the loop is formed from a tubular material.

42. The bone surgical apparatus of claim 36 further comprising:
a fastener attached to the second end section of the rod wherein the fastener comprises a head including spaced apart walls defining a recess for receiving an alignment rod.

43. The bone surgical apparatus of claim 33 further comprising:
a fastener attached to the rod between the first end section and the second end section, wherein the fastener comprises a head including spaced apart walls defining a recess for receiving an alignment rod.

44. The bone surgical apparatus of claim 33 wherein:
the first end section of the rod and the second end section of the rod are connected by a hinge.

45. The bone surgical apparatus of claim 44 wherein:
a first section of the hinge is attached to the first end section of the rod,
a second section of the hinge is attached to the second end section of the rod, and
the hinge includes a locking device for locking the hinge in one of a plurality of relative positions between the first section of the hinge and the second section of the hinge.

46. The bone surgical apparatus of claim 33 wherein:
the first end section of the rod and the second end section of the rod are connected by an adjustment mechanism structured to retain the first end section of the rod and the second end section of the rod at any of a plurality of distances between the first end section of the rod and the second end section of the rod thereby providing for variable lengths of the rod.

47. The bone surgical apparatus of claim 46 wherein:
the adjustment mechanism is connected by a hinge to at least one of the first end section of the rod and the second end section of the rod.

48. A bone surgical apparatus for manipulating a curvature of a spine, the bone surgical apparatus comprising:
a latch connector comprising a rod adapter dimensioned to attach to a fusion rod, a central section attached to the rod adapter wherein the central section is dimensioned to attach to a head of a pedicle screw, and a latch attached to the central section wherein the latch comprises a first wall and a second wall defining an interior region for receiving a portion of a loop.

49. The bone surgical apparatus of claim 48 wherein:
the first wall has a first terminal end,
the second wall has a second terminal end,
the first terminal end and the second terminal end are positioned to define a space between the first terminal end and the second terminal end,
the latch includes a movable barrier for opening and closing the space between the first terminal end and the second terminal end,
the portion of the loop is received in the interior region when the space is open, and
the portion of the loop is retained in the interior region when the space is closed.

50. The bone surgical apparatus of claim 48 further comprising:
a fusion rod;
a first connector comprising a first rod having a first end section and a second end section wherein the first rod is dimensioned to connect a pair of pedicle screws, and a first fastener attached to the first rod wherein the first fastener comprises a first head including spaced apart walls defining a first recess, the fusion rod being positioned in the first recess and being connected to the first head;
a first pair of pedicle screws configured to attach to a first vertebra, the first pair of pedicle screws being attached to the first rod of the first connector;
a second connector comprising a second rod having a first end section and a second end section wherein the second rod is dimensioned to connect a pair of pedicle screws, and a second fastener attached to the second rod wherein the second fastener comprises a second head including spaced apart walls defining a second recess, the fusion rod being positioned in the second recess and being connected to the second head;
a second pair of pedicle screws configured to attach to a second vertebra, the second pair of pedicle screws being attached to the second rod of the second connector;
a third connector a third rod having a first end section and a second end section wherein the third rod is dimensioned to connect a pair of pedicle screws, and a second latch attached to the first end section of the third rod wherein the second latch comprises a first wall and a second wall defining an interior region for receiving a portion of the loop;
a third pair of pedicle screws configured to attach to a third vertebra, the third pair of pedicle screws being attached to the third rod of the third connector; and
a loop having a first portion retained in the interior region of the latch and a second portion retained in the interior region of the second latch,
wherein the rod adapter of the latch connector is attached to the fusion rod, and the central section of the latch connector is attached to the second head of the second fastener.

51. The bone surgical apparatus of claim 50 wherein:
the rod adapter of the latch connector is attached to the fusion rod by a set screw.

52. The bone surgical apparatus of claim 50 wherein:
the central section of the latch connector surrounds the second head of the second fastener to engage the second head of the second fastener when the latch connector is attached to the second head of the second fastener.

53. A bone surgical apparatus for manipulating a curvature of a spine, the bone surgical apparatus comprising:
an adjustment mechanism comprising a first rod section having an inner end and an outer end, a second rod section having an inner end and an outer end having an outer surface shape, and a hinge attached to the inner end of the first rod section and to the inner end of the second rod section,
wherein the outer end of the first rod section comprises a wall defining a recess dimensioned for receiving a rod end section having a perimeter shaped the same as the outer surface shape of the outer end of the second rod section.

54. The bone surgical apparatus of claim 53 wherein:
a first section of the hinge is attached to the inner end of the first rod section,
a second section of the hinge is attached to the inner end of the second rod section, and
the hinge includes a locking device for locking the hinge in one of a plurality of relative positions between the first section of the hinge and the second section of the hinge.

55. The bone surgical apparatus of claim 54 further comprising:
a second adjustment mechanism comprising a third rod section having an inner end and an outer end, a fourth rod section having an inner end and an outer end having an outer surface shape, and a second hinge attached to the inner end of the third rod section and to the inner end of the fourth rod section,
wherein the outer end of the third rod section comprises a wall defining a recess dimensioned for receiving the outer end of the second rod section.

56. The bone surgical apparatus of claim 53 wherein:
the hinge includes a ratchet.

57. The bone surgical apparatus of claim 55 further comprising:
a first pedicle screw configured to attach to a first vertebra, the first rod section being attached to the first pedicle screw; and
a second pedicle screw configured to attach to a second vertebra, the fourth rod section being attached to the second pedicle screw,
wherein the outer end of the second rod section is received in the recess in the outer end of the third rod section.

58. The bone surgical apparatus of claim 57 wherein:
the outer end of the second rod section is secured in the recess in the outer end of the third rod section by a set screw.

59. The bone surgical apparatus of claim 57 wherein:
the outer end of the second rod section is secured in the recess in the outer end of the third rod section by an interference fit.

60. The bone surgical apparatus of claim 57 wherein:
a first section of the second hinge is attached to the inner end of the third rod section,
a second section of the second hinge is attached to the inner end of the fourth rod section, and
the second hinge includes a locking device for locking the second hinge in one of a plurality of relative positions of the second hinge between the first section of the second hinge and the second section of the second hinge.

61. The bone surgical apparatus of claim 60 wherein:
the hinge is locked in one of the plurality of relative positions of the hinge by a first set screw, and
the second hinge is locked in one of the plurality of relative positions of the second hinge by a second set screw.

62. The bone surgical apparatus of claim 53 further comprising:
a fusion rod;
a first pedicle screw configured to attach to a first vertebra, the fusion rod being attached to the first pedicle screw;
a second pedicle screw configured to attach to a second vertebra, the second rod section being attached to the second pedicle screw,
wherein an end of the fusion rod is received in the recess in the outer end of the first rod section.

63. The bone surgical apparatus of claim 62 wherein:
the end of the fusion rod is secured in the recess in the outer end of the first rod section by a set screw.

64. A bone surgical apparatus for manipulating a curvature of a spine, the bone surgical apparatus comprising:
a first pedicle screw and a second pedicle screw configured to attach to a first vertebra;
a third pedicle screw and a fourth pedicle screw configured to attach to a second vertebra; and
a fixator having a first end section and a second end section, the first end section of the fixator including a hinged rod attached to the first pedicle screw, and the second end section of the fixator being attached to the fourth pedicle screw.

65. The bone surgical apparatus of claim 64 wherein:
a first end of the hinged end is received in a recess in a head of the first pedicle screw for attachment to the first pedicle screw,
a second end of the hinged end is received in a recess in a head of the second pedicle screw for attachment to the second pedicle screw and
the second end section of the fixator is received in a recess in a head of the fourth pedicle screw for attachment to the fourth pedicle screw.

66. A method for manipulating a curvature of a spine of a subject, the method comprising:
surgically implanting a bone surgical apparatus of claim 60 into the subject such that the first pedicle screw and the second pedicle screw are attached to the first vertebra and the third pedicle screw and the fourth pedicle screw are attached to the second vertebra;
moving the fixator to achieve a desired alignment of the first vertebra and the second vertebra;
removing the hinged end from the recess in the head of the first pedicle screw; and
attaching an alignment rod to the first pedicle screw and the third pedicle screw.

67. A method for manipulating a curvature of a spine of a subject, the method comprising:
surgically implanting a bone surgical apparatus of claim 26 into the subject such that the first pedicle screw and the second pedicle screw are attached to the first vertebra and the third pedicle screw and the fourth pedicle screw are attached to the second vertebra;
moving the fixator to achieve a desired alignment of the first vertebra and the second vertebra;
removing the connector from the first pedicle screw; and
attaching an alignment rod to the first pedicle screw and the third pedicle screw.
